(19) United States
(12) Patent Application Publication
(10) Pub. No.: US 2022/0344018 A1
(43) Pub. Date: Oct. 27, 2022

(54) Machine Learning Derived Multimorbidity Risk Scores for Generalizable Patient Populations

(71) Applicant: Anthem, Inc., Indianapolis, IN (US)

(72) Inventors: Theodore Goldstein, Los Altos, CA (US); Bobby Samuel, Redwood City, CA (US); Beau Norgeot, Los Gatos, CA (US); Abhishaike Mahajan, Austin, TX (US); David Blankley, Manakin Sabot, VA (US)

(21) Appl. No.: 17/237,591

(22) Filed: Apr. 22, 2021

(51) Int. Cl. G16H 20/00; G16H 50/30; G16H 50/20

(52) U.S. Cl. CPC G16H 20/00; G16H 50/30; G16H 50/20

(57) ABSTRACT

A system and method for generating health care plans for patients are provided. The method includes extracting data items from age-agnostic medical claims data for a plurality of patients. The method also includes, for each health condition of a plurality of health conditions, aggregating one or more of the data items into one or more feature sets based at least on a data item type and a set of rules, and applying one or more machine learning models to the one or more feature sets to predict a respective risk score for the respective health condition for a respective patient. The method also includes computing a total health score based on the predicted respective risk score for each health condition for the respective patient. The method subsequently generates a report that indicates a health care plan for the respective patient based on the total health score in relation to a particular age group.

### TECHNICAL FIELD

The disclosed implementations relate generally to healthcare applications and more specifically to a method, system, and device for machine learning derived multimorbidity risk scores for healthcare.

### BACKGROUND

Health assessments and clinical risk score calculations are an important part of primary clinical care and provide a snapshot of a patient's health status and health risks. In addition to health assessment tools, computable risk scores tools can be used to assess patient health status for specific conditions. Risk scores can help identify specific interventions to benefit patients, and provide actionable information to guide tests and medications. Multimorbidity risk scores, which factor in the presence of several chronic conditions, can provide insights into general morbidity and mortality. Examples of multimorbidity scores include the Charlson Index, Elixhauser Index, Adjusted Clinical Groups System, Chronic Disease Score, and the Duke Severity of Illness. In general, the number of co-occurring medical conditions is associated with increased adverse medical outcomes as well as the increased use of medical services. This is particularly true for older individuals since the number of co-occurring medical conditions will increase with age. Conventional methods that develop and assess the quality of risk scores, include condition-specific risk scores and multimorbidity scores that typically suffer from various limitations. For instance, the GRASP framework assesses risk scores based on the target population, internal or external validation, potential effects, and usability which vary widely across different scores. Aside from risk scores, several laboratory measurement-based risk models (using regression techniques and machine learning approaches) have been developed to predict the presence or severity of specific conditions. Obtaining a snapshot of patient health frequently involves integrating several different sources and thoroughly reviewing diagnostic, procedure, prescription, and laboratory data. This integration process is non-trivial: interpreting various disease-specific and diagnosis-derived multimorbidity risk scores can result in an incomplete, patchwork profile of a patient's health, and information can be missed during chart reviews. Currently, there is no unified, integrated risk score model that incorporates diagnostic, procedural, prescription, and laboratory data into a comprehensible single score or set of scores that reflects the clinical risk of an adverse outcome irrespective of age, and derived from a large, statistically-powered, representative population of patients.

### SUMMARY

Accordingly, there is a need for a total health profile, a set of machine-learning derived measures of an individual's comprehensive clinical risk. The total health profile presents clinical risk in five separate models (sometimes called “Component Scores”, or CS) producing risk-scores specific to cardiovascular (“heart score”), respiratory (“lung score”), neuropsychiatric (“neuro score”), renal (“kidney score”), and gastrointestinal (“digestive score”) conditions, according to some implementations. From these Component Scores, some implementations derive a total health score (sometimes called THS), a single, multimorbid, and unified view of a patient's overall health risks across the Component Scores. In some implementations, each of these six scores are independently modeled using medical claims data consisting of demographic information, diagnostic codes, laboratory results, prescriptions, and medical procedural data. Each scores' estimate of clinical risk represents the likelihood of score-related inpatient hospital visits over a future time period (e.g., the next 24 months). Inpatient visits are known to correlate with the number of morbidities and the general health of an individual. After training, testing, and calibrating the THS and the five organ-system specific CS, some implementations analyze the properties of each score and their intercorrelations for further tuning. Subsequently, some implementations post-process the THS and component scores to visualize data for easy interpretation and/or to inform patient care.

In one aspect, some implementations include a computer-implemented method of generating health care plans for patients. The method is executed at a computing device coupled to one or more memory units each operable to store at least one program. One or more servers having at least one processor communicatively coupled to the one or more memory units, in which the at least one program, when executed by the at least one processor, causes the at least one processor to perform the method.

The method includes extracting data items from age-agnostic medical claims data for a plurality of patients. The method also includes, for each organ-system-specific health condition of a plurality of organ-system-specific health conditions for a respective patient: (i) aggregating one or more of the data items into one or more feature sets based at least on a data item type and a set of rules, and (ii) applying one or more machine learning models to the one or more feature sets to predict a respective risk score for the respective health condition for a respective patient. In some implementations, the one or more machine learning models were previously trained by performing risk classification analysis on the data items from the age-agnostic medical claims data for the plurality of patients to calculate organ-system-specific risk score representing health risks for a specific organ-system. The method also includes computing a total health score based on the predicted respective risk score for each health condition for the respective patient. For example, the total health score is calculated independently from the predicted specific organ-system scores, and has the boolean sum of the labels of the predicted specific organ-system scores (sometimes called component scores or CS). For example, if the heart CS label is 1, the total health score (sometimes called THS) label will also be 1. In some implementations, the minimum of all CS scores is nearly equivalent to the THS, as the THS is supposed to reflect all risks.

The method also includes generating a report that indicates a health care plan for the respective patient based on the total health score in relation to a particular age group.

In some implementations, the respective risk score represents the likelihood of inpatient hospital visits over a predetermined future time period for the respective health condition.

In some implementations, the one or more machine learning models include a respective machine learning model for each health condition of the plurality of health conditions. The method further includes applying the respective machine learning model for the respective health condition to the one or more feature sets to predict the respective risk score for the respective health condition for a respective patient.

In some implementations, the plurality of health conditions includes cardiovascular, respiratory, neuropsychiatric, renal, and gastrointestinal conditions.

In some implementations, the medical claims data includes demographic information, diagnostic codes, laboratory results, prescriptions, and medical procedural data.

In some implementations, the one or more machine learning models include a respective gradient boosted classifier for each health condition. In some implementations, the method further includes aggregating the one or more of the data items into one or more feature sets further based on selecting a predetermined number of features of the respective gradient boosted classifier for the respective health condition. In some implementations, the predetermined number of features includes number of inpatient hospital visitations during the data-collection period.

In some implementations, the method further includes performing steps of inversion, scaling to 0-100, and normalization by age, on the respective score, for generating the report. In some implementations, the one or more machine learning models includes a gradient-boosted tree model that outputs calibrated likelihoods of an inpatient visitation between [0, 1], where 1 represents a 100% chance that a patient will have an inpatient visitation during a predetermined follow-up period, and wherein the inversion comprises subtracting the likelihood from 1, scaling includes multiplying result of the inversion by 100, and normalization by age includes calculating percentile amongst patients of a predetermined age group.

In some implementations, the method further includes calculating correlation between the respective score for each health condition and the total health score, while generating the report.

In some implementations, the one or more machine learning models include a gradient-boosted tree classifier that is trained using a training dataset that includes diagnoses, laboratory values, procedures, and prescription data as inputs and inpatient visits as binary labels, and calibrated using an isotonic regression with 3-fold cross-validation over the training dataset.

In some implementations, generating the report includes displaying the total health score and a breakdown of the total health score in terms of the respective score for each health condition, a comparison of the total health score of the respective patient to other patients in same age group as the respective patient, vitals, and/or data used to compute the total health score, in addition to a health care plan for alleviating at least some of the health conditions.

In another aspect, some implementations include a system configured to perform any of the methods described herein.

### BRIEF DESCRIPTION OF THE DRAWINGS

For a better understanding of the various described implementations, reference should be made to the Description of Implementations below, in conjunction with the following drawings in which like reference numerals refer to corresponding parts throughout the figures.

FIG. 1 shows a schematic diagram of a system 100 for calculating multimorbidity risk scores for generalizable patient populations, according to some implementations.

FIG. 2A shows a table with example demographic profile of patients included in an analysis cohort, and FIG. 2B shows a table with example zip-code demographic profile of patients included in the analysis cohort, according to some implementations.

FIG. 3 shows a bar chart that indicates positive correlation between percentage of patients with inpatient visits and age of the patient, according to some implementations.

FIG. 4A shows a table of discriminative and calibration metrics for each score in a large test set, according to some implementations.

FIG. 4B shows Pearson R correlations between all predicted scores, for the table shown in FIG. 4A, according to some implementations.

FIG. 5 shows ROC curves that plots true positive rate against false positive rate, for each risk score, according to some implementations.

FIG. 6A—shows an example calibration curve for a heart risk model, according to some implementations.

FIG. 6B shows an example calibration curve for a lung risk model, according to some implementations.

FIG. 6C shows an example calibration curve for a neuro risk model, according to some implementations.

FIG. 6D shows an example calibration curve for a kidney risk model, according to some implementations.

FIG. 6E shows an example calibration curve for a digestive risk model, according to some implementations.

FIG. 6F shows an example calibration curve for a total health risk score model, according to some implementations.

FIGS. 7A-7C show results of sensitivity analysis, according to some implementations.

FIG. 8 shows a table with a list of laboratory results or physiological measurements or vitals used in calculation of each risk score, according to some implementations.

FIG. 9 shows an example list of CPT codes used to identify inpatient visits, according to some implementations.

FIG. 10 shows a table of chronic conditions used to derive input for each risk score, according to some implementations.

FIGS. 11A-11D show a table of acute ICD Codes, stratified by CS/THS models, according to some implementations.

FIGS. 12A-12D show a table of Generic product identifier (GPI) codes corresponding to antihypertensives, glucose-lowering, lipid-lowering, and antithrombotic medications, according to some implementations.

FIG. 13 shows plots of precision-recall curve for health scores on a test-set, according to some implementations.

FIG. 14 shows a table of baseline, logistic regression metric metrics for each score in a test set, according to some implementations.

FIG. 15 shows a table of positive and negative label counts corresponding to each of the individual scores for a large data set, according to some implementations.

FIG. 16 shows split violin plots for predicted, calibrated scores of age-stratified, healthy/unhealthy patients, according to some implementations.

heart, lung, neuro, kidney, digestive, and Total-Health risk scores.

FIG. 17A shows feature importance for heart risk score model, according to some implementations.

FIG. 17B shows feature importance for a lung risk score model, according to some implementations.

FIG. 17C shows feature importance for a neuro risk score model, according to some implementations.

FIG. 17D shows feature importance for a kidney risk score model, according to some implementations.

FIG. 17E shows feature importance for a digestive risk score model, according to some implementations.

FIG. 17F shows feature importance for a total-health risk score model, according to some implementations.

FIGS. 18A-18F depicts violin plots for predicted scores that have been post-processed to represent percentile of score within an age decade bin.

FIG. 19 depicts as interface with health breakdown, in some implementations.

FIGS. 20A and 20B depicts variations across decade age-groups and sexes for each of the six risk scores, according to some implementations.

### DESCRIPTION OF IMPLEMENTATIONS

Reference will now be made in detail to implementations, examples of which are illustrated in the accompanying drawings. In the following detailed description, numerous specific details are set forth in order to provide a thorough understanding of the various described implementations. However, it will be apparent to one of ordinary skill in the art that the various described implementations may be practiced without these specific details. In other instances, well-known methods, procedures, components, circuits, and networks have not been described in detail so as not to unnecessarily obscure aspects of the implementations.

It will also be understood that, although the terms first, second, etc. are, in some instances, used herein to describe various elements, these elements should not be limited by these terms. These terms are only used to distinguish one element from another. For example, a first electronic device could be termed a second electronic device, and, similarly, a second electronic device could be termed a first electronic device, without departing from the scope of the various described implementations. The first electronic device and the second electronic device are both electronic devices, but they are not necessarily the same electronic device.

The terminology used in the description of the various described implementations herein is for the purpose of describing particular implementations only and is not intended to be limiting. As used in the description of the various described implementations and the appended claims, the singular forms “a”, “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “includes,” “including,” “comprises,” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” is, optionally, construed to mean “when” or “upon” or “in response to determining” or “in response to detecting” or “in accordance with a determination that,” depending on the context. Similarly, the phrase “if it is determined” or “if [a stated condition or event] is detected” is, optionally, construed to mean “upon determining” or “in response to determining” or “upon detecting [the stated condition or event]” or “in response to detecting [the stated condition or event]” or “in accordance with a determination that [a stated condition or event] is detected,” depending on the context.

As described above in the Summary section, there is a need for an automated, machine-learning-derived multi-morbidity risk profile for acute clinical events that can be used for individualized patient care management and patient education, at scale and continuously adjusted. This task is challenging because risk is an abstract concept, and there are no one good, ground-truth for calculating it. Some implementations determine which directly obtainable data sources would serve as the most useful proxies for comprehensive patient health and risk. In some implementations, as described below, this score is calculated automatically and at scale, without relying on clinician time/effort. Conventional scoring techniques, on the other hand, generally require patient behavior and/or familial history as input. In order to facilitate clinical decision making, as described below, some implementations create a profile that could be broken apart into organ-system-specific component scores, such as cardiovascular, respiratory, neuropsychiatric, renal, and gastrointestinal health. The profile is easily extended to include other organ systems.

Clinical risk scores are scalar values that measure a patients risk for a certain clinical outcome. Such scores have been used in clinical practice a long time, serving as useful ways for doctors to quickly ascertain patient risk for a certain condition (e.g., diabetes), procedural outcome (e.g., ER visit), and also as a healthcare education tool for patients to understand their own health status. Conventional risk scores, such as Framingham, are suboptimal for a variety of reasons. Multimorbidity is often extremely important when considering a patients risk for any single morbidity, but typical clinical risk scores usually only focus on one comorbidity. Of the multimorbidity scores that exist, they cannot be broken apart to reflect single-comorbidity risks. Moreover, these scores often overly rely on diagnoses or procedures (disregarding prescriptions and lab values), and often require behavior and familial history data, which can be difficult to collect when scaling risk scores to millions of patients. Finally, none of these scores claim to measure the abstract idea of general health.

In some implementations, the total health score is an interpretable, calibrated multimorbidity risk score that can be further broken down into cardiovascular, respiratory, neuropsychiatric, renal, and gastrointestinal risk scores. This allows the systems to comprehensively quantify patient health from their overall health to organ-system-specific health. In some implementations, the system calculates the scores passively and automatically using a machine-learning model trained on data from a patient's Electronic Health Record and requires no additional user input as to their behavior, familial history, and genetics.

Some implementations generate a clinical total health score and component organ scores. Some implementations group raw clinical data so as to get a data-driven aggregate view of health from the collection of a patient's clinical events, which is somewhat conceptually analogous in utility to generating a credit score from the collection of person's financial transactions. The total health score has additional advantages over a credit score in that a score of 80 (out of 100) is directly interpretable clinically (unlike a credit score of 720), and the score is designed in such a way that the actions necessary to improve a score are clear and follow clinical best practices.

Some implementations include an automated method to calculate the scores. Some implementations include a machine learning system that generates the scores and continuously monitors health data and processes the data, updating scores for individuals constantly and using more data when the data becomes available from each individual to generate more precise scores for every individual. In some implementations, every clinical visit would result in an updated score (though not an updated model). In some implementations, an updated model is generated once a year, and would largely just be composed of making a brand new model on the extra year's worth of data. In some implementations, the techniques described herein are extended to include additional types of data, such as data from sensors on wearables that track activity. Examples of such data include PPG signals, ECG signals, respiratory rate, and heart-rate. In some implementations, static variables, such as frequency or average amplitude, are derived from these bio-signals and then input into the models as extra variables. In some implementations, the system is also designed in a way that it can be extended to include additional organ systems or health components.

Some implementations split the risk scores into organ-system risk scores. Some implementations automatically collect healthcare information. Some implementations use manually input data or augmented data. Some implementations provide clinical guidelines alongside a risk score. Some implementations quantify health, rather than just the risk of developing a medical condition. Some implementations predict general health via risk of an inpatient hospital visit related to a medical condition. Unlike conventional systems that only provide the probability that a patient may develop Type 2 Diabetes in the next year, the total health score indicates how large an impact a patient's health conditions are likely to have on the patient's overall quality of life, and additionally provide how each part of the patients' health contributes to that impact (e.g., each component score is shown independently), and what the patient can do to improve health. The techniques described herein can be used to derive a unified multi-morbid set of clinical risk scores that cover most pathophysiologies, using tabular clinical information of a patient for its calculation, based on a configurable definition of what clinical risk means in a given context (e.g., a patient cohort, within a geographic region, in a demography, etc.), and provide an immediate clinical interpretation. Conventional clinical risk scores are specific to a single group of conditions, and combining the risk scores could potentially result in a patch-work understanding of patient health. Having a unified risk score of overall patient clinical risk, alongside condition-specific risk scores, is likely very useful for clinicians.

### Patient Cohort

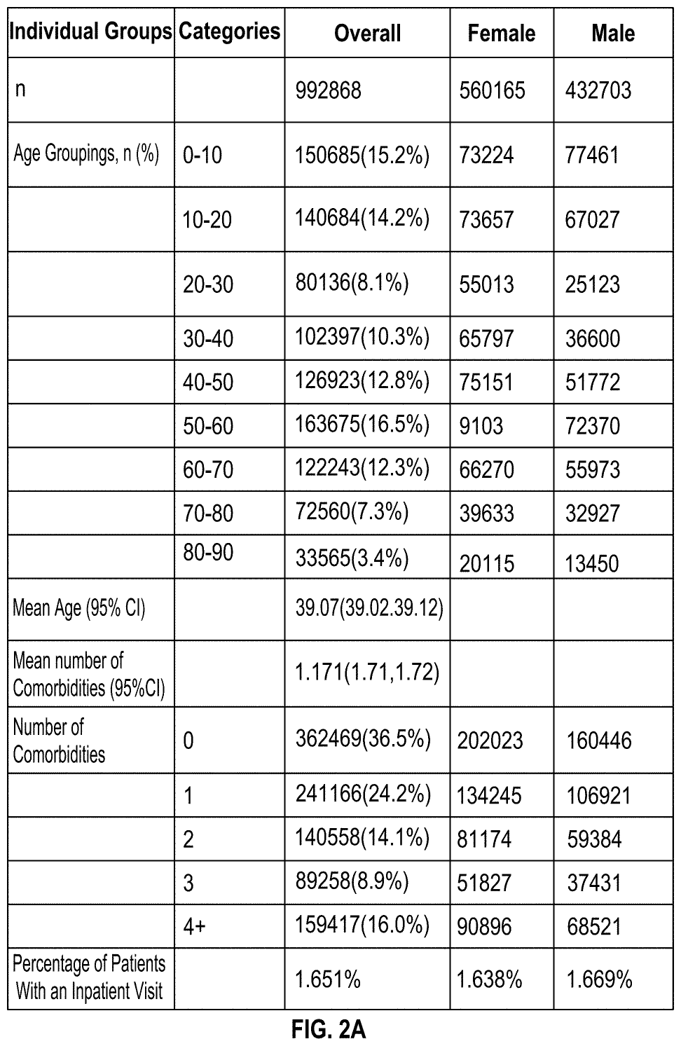

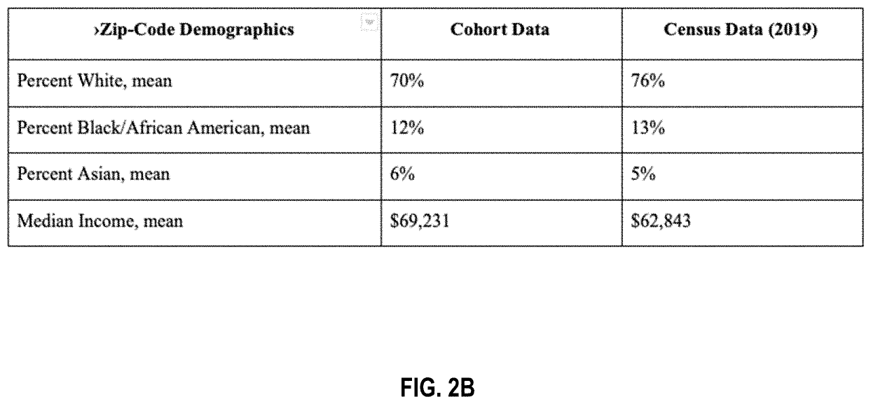

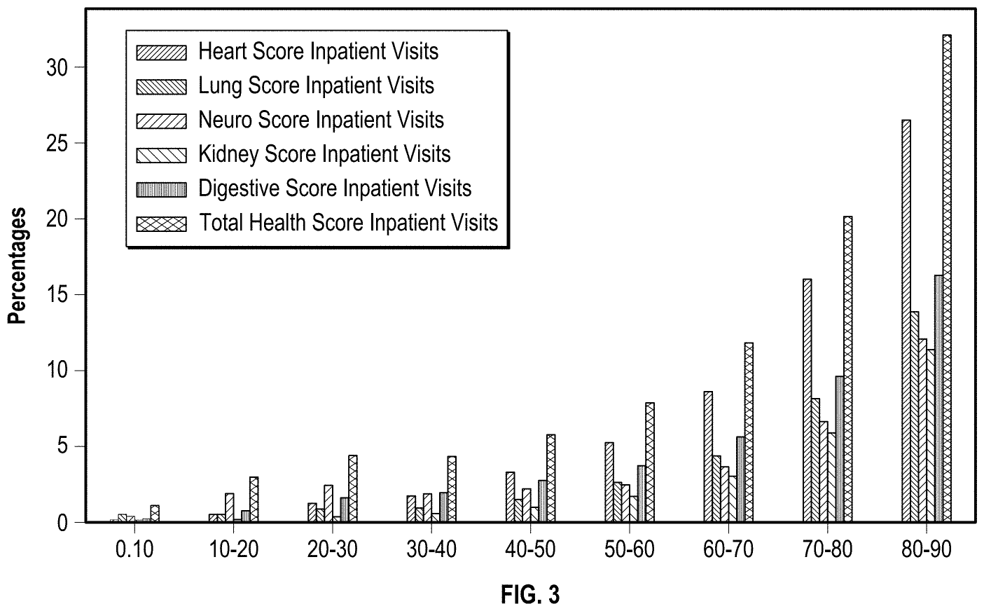

FIG. 2A shows a table with demographics of an example retrospective cohort. The analysis cohort includes 992,868 patients, the majority of whom were female (56.4%), matching US Census data. The median age of the cohort was 41, which was higher than the national average. Additionally, the number of comorbidities tended to increase with age, consistent with previous findings. FIG. 2B shows a summary of zip-code level demographics for the analysis cohort. The population was 70% white, lower than the national reported rate of 76%, 6% Asian, higher than the national reported rate of 5% in the current US Census, and 12% African American, lower than the nationally reported rate of 13% in the current US Census, while median income was nearly identical ($69,231 versus $62,843 from the census). FIG. 3 illustrates the positive correlation of inpatient visits (used as a measure of clinical risk) with age, which is concordant with previous studies.

### Model Performance and Validation

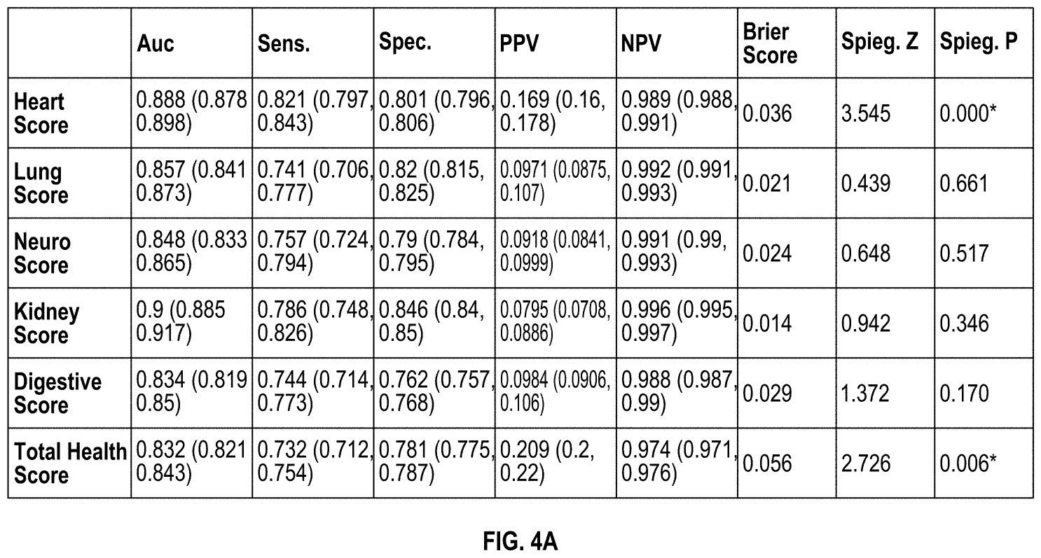

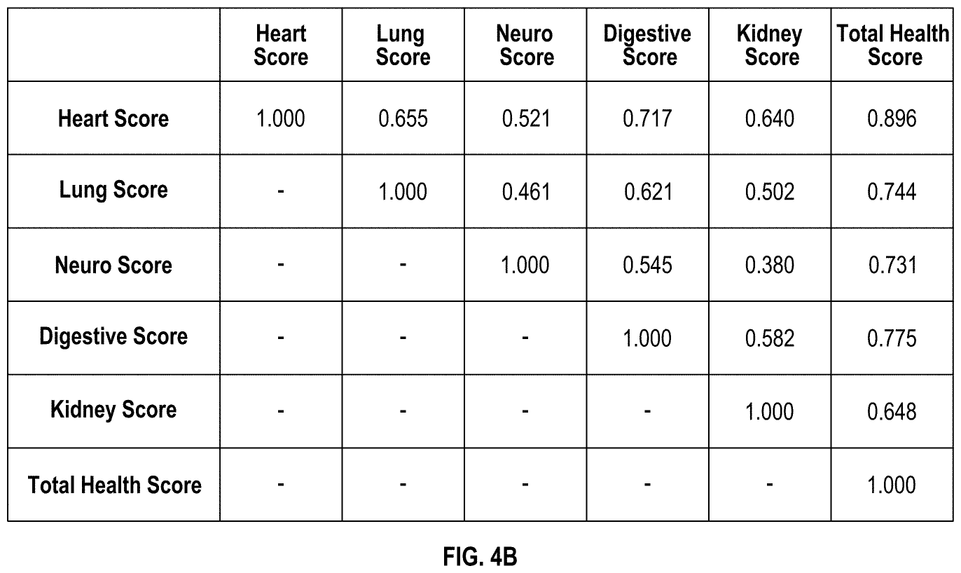

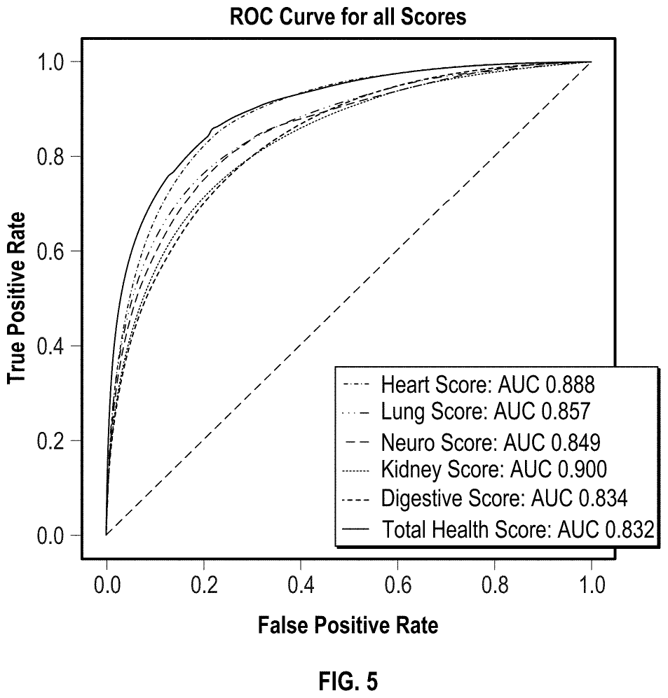

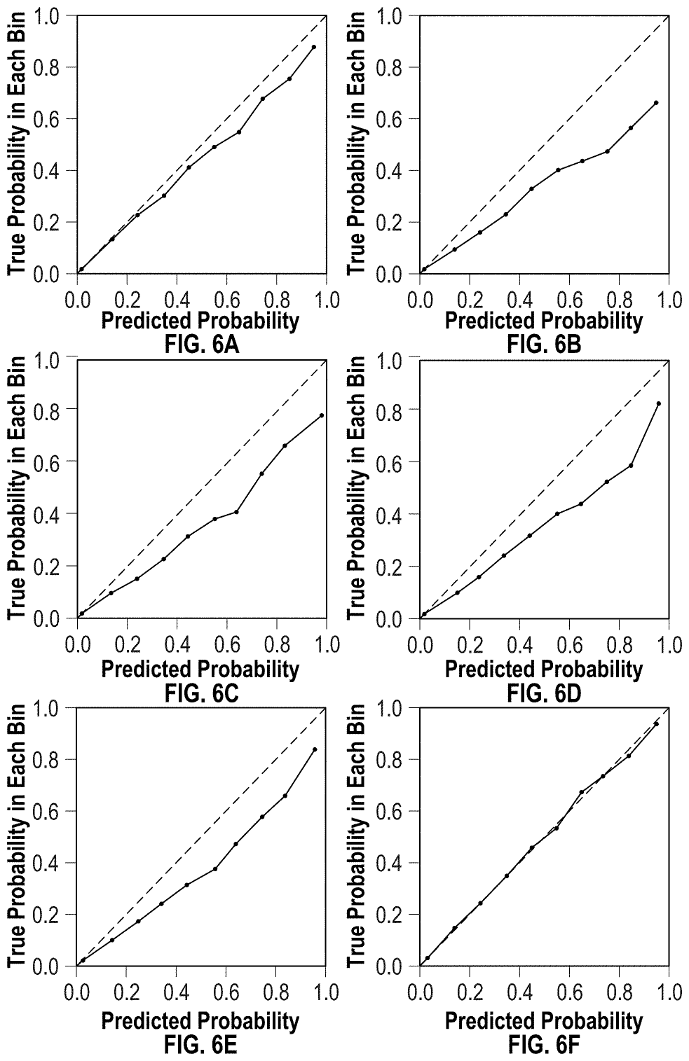

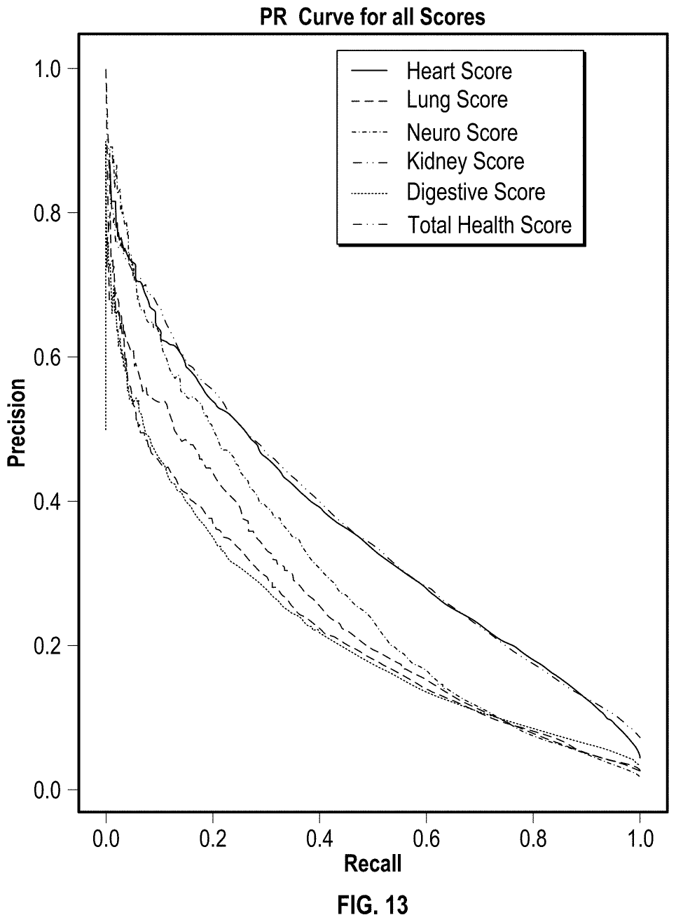

FIG. 5 shows a graph plot that assesses ROC-AUC of THS and component score models, and calculates the sensitivity, specificity, and positive and negative predictive value of each score model in predicting a calibrated probability of clinical events (specified by the “score label” or organ system category, such as cardiovascular or respiratory health) occurring during a 2-year follow-up period shown in the table in FIG. 4A, according to some implementations. As shown, all AUCs are above 0.83, the highest being the cardiovascular component score AUC of 0.888. The sensitivities and specificities for all models were in the range between 0.7-0.8. FIG. 13 shows a graph plot of the precision-recall curve for all scores on the test-set. The graph plot was used to assess calibration using Brier Scores and Spiegelhalter's Z-score for each model (FIG. 4A), which established that four out of the six models met this criteria for being well-calibrated, except for the THS and the cardiovascular component score. The calibration curves for all models (shown in FIG. 6) indicated that the THS model was well calibrated, with a small degree of underprediction in all other component scores. The THS and component scores were then analyzed by plotting the distribution of scores as a function of age and general health (measured by presence of pre-existing comorbidities).

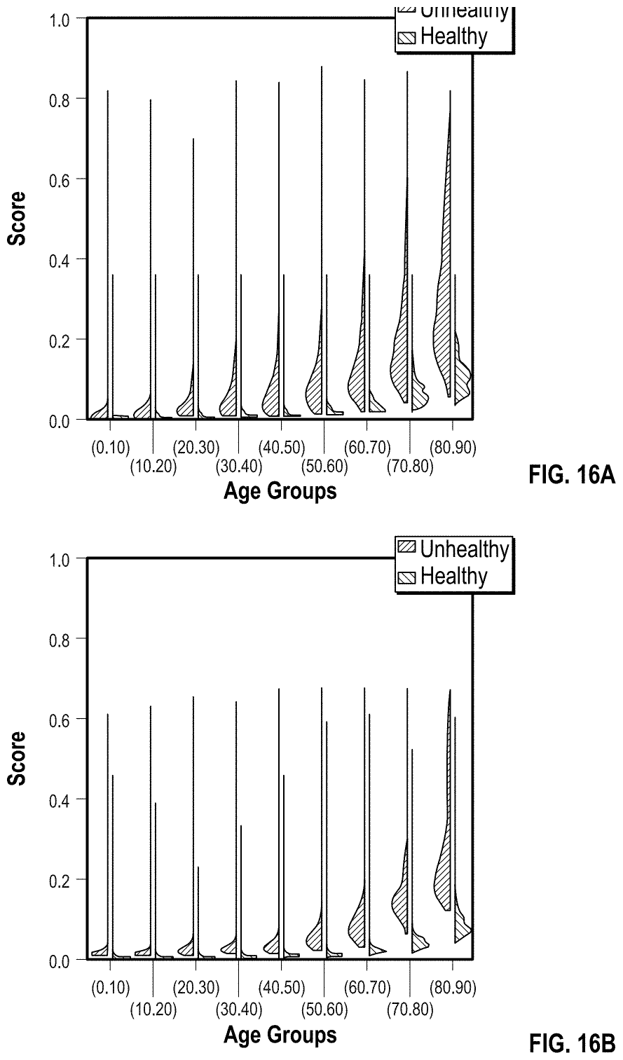

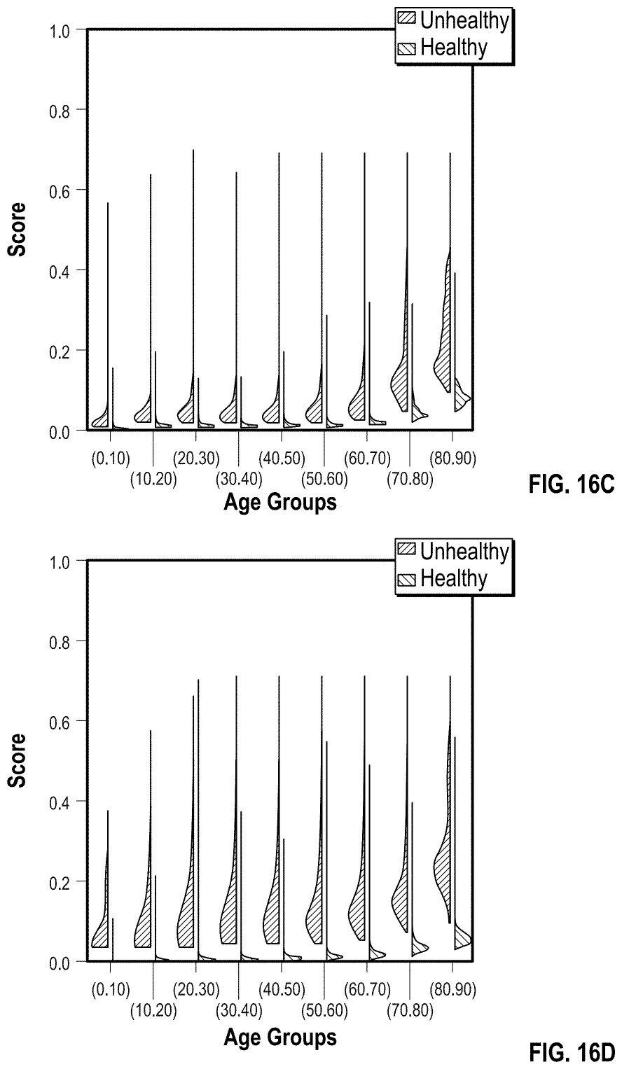

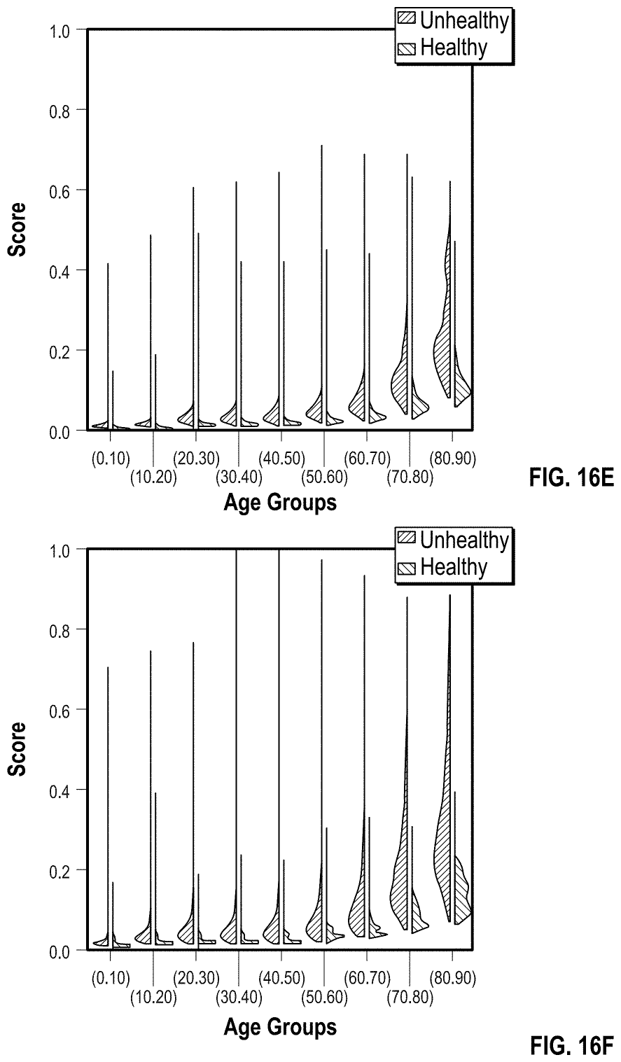

The distributions of the THS and the component scores among various age groups for healthy patients with no comorbidities and unhealthy patients with at least one Elixhauser comorbidity related to the given component, were plotted and analyzed as shown in FIG. 16. The THS and component scores were tightly distributed, and were very low but monotonically increased slightly with age for healthy patients. As expected, the scores for unhealthy patients were centered higher, had significantly higher variance, and increased monotonically at a higher rate. FIG. 16 shows split violin plots for predicted, calibrated scores of age-stratified, healthy/unhealthy patients. Healthy is defined as having no comorbidities related to that score. Unhealthy is defined as having 1 or more comorbidities related to that score (defined in the appendix). A-F refers to, in order, heart, lung, neuro, kidney, digestive, and Total-Health risk scores.

To further inspect the results of the models, the intercorrelations between the various model scores were calculated using Pearson's R (shown in FIG. 4B). As expected, the correlation matrix indicated that the models were generally highly correlated, with correlation values between the component scores and the THS being in the range of 0.65-0.90. The highest correlations were noted for the cardiovascular component score and the THS, while the lowest correlation was noted with the renal score.

### Baseline

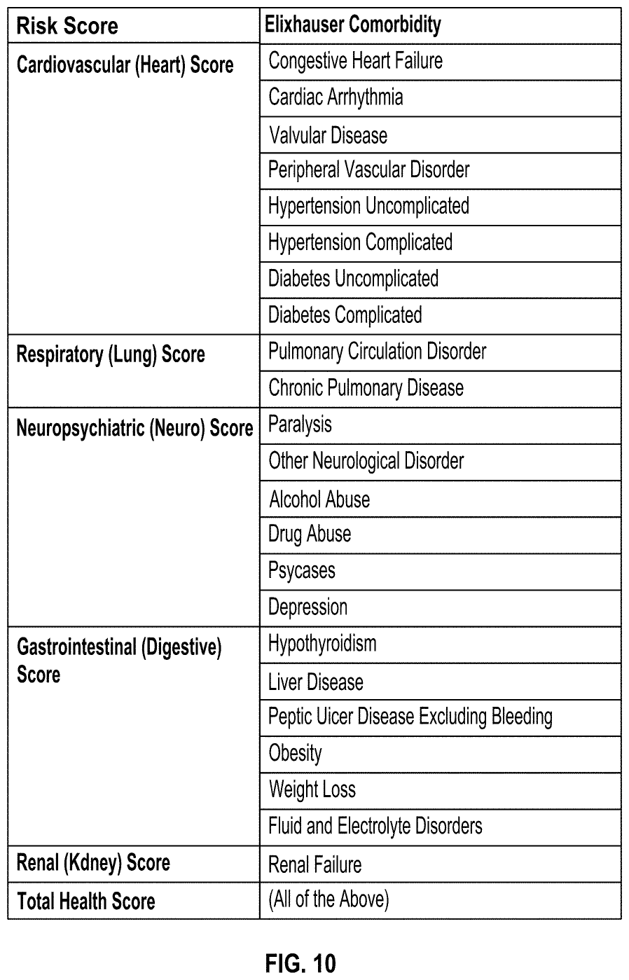

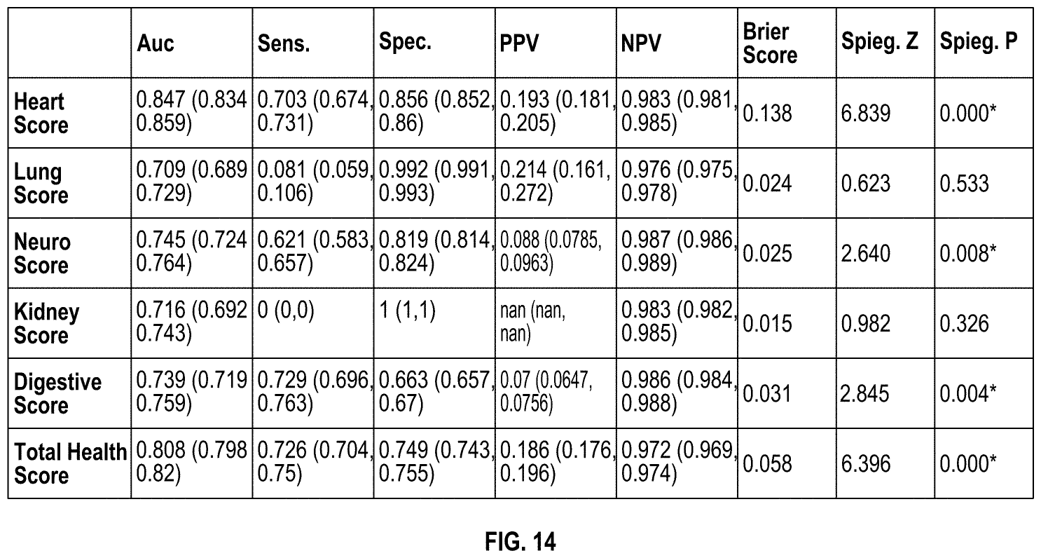

A simplified feature set was fit to the above discussed labels and otherwise identical models, in order to establish a baseline for all six scoring models. The simplified feature set is limited to binary Elixhauser comorbidities, filtered to only the relevant ones for a given component score model (mappings shown in FIG. 10). The metrics of each baseline model is shown in FIG. 14. These baseline models score consistently worse in AUC and sensitivity, but perform comparably to the trained models in calibration metrics.

### Sensitivity Analysis

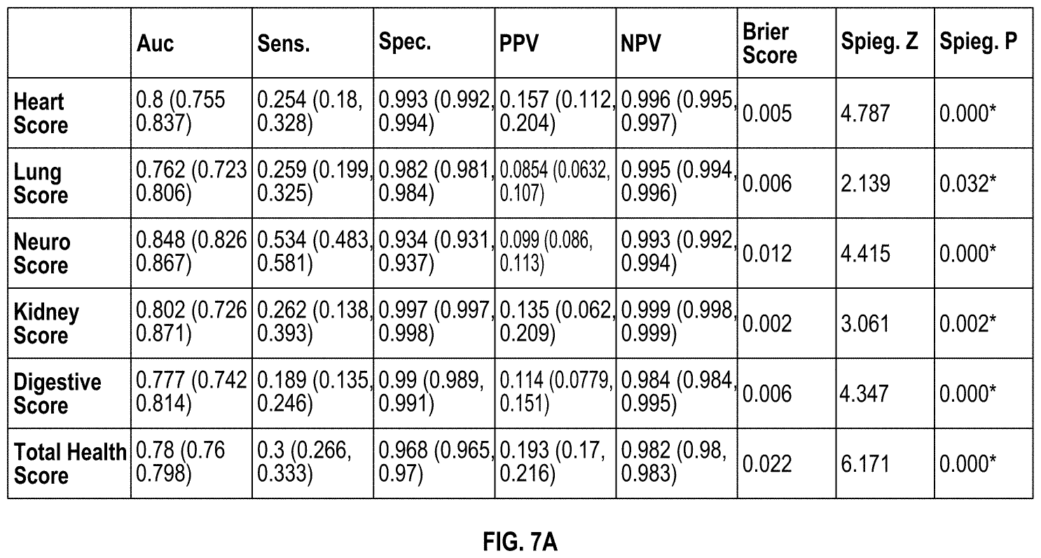

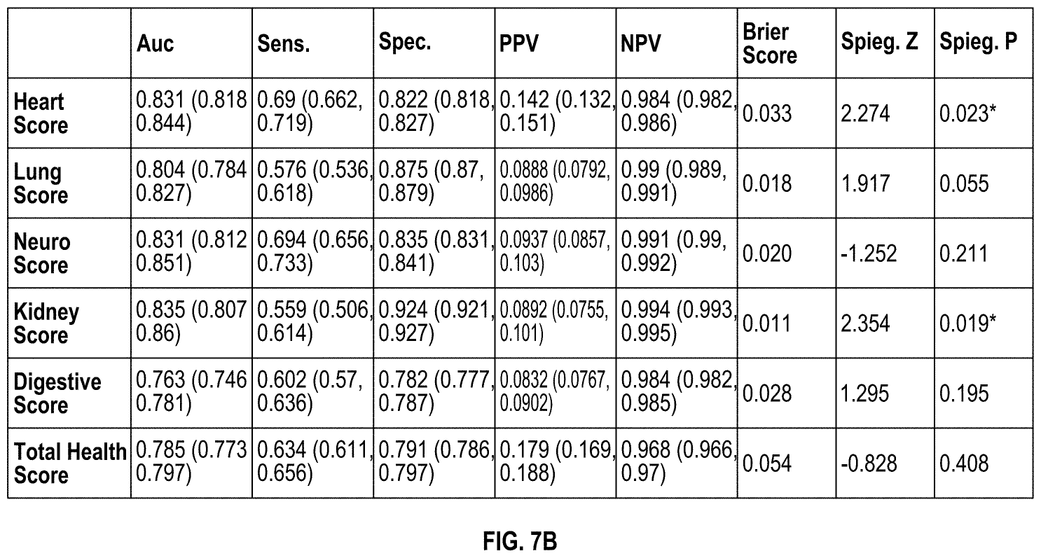

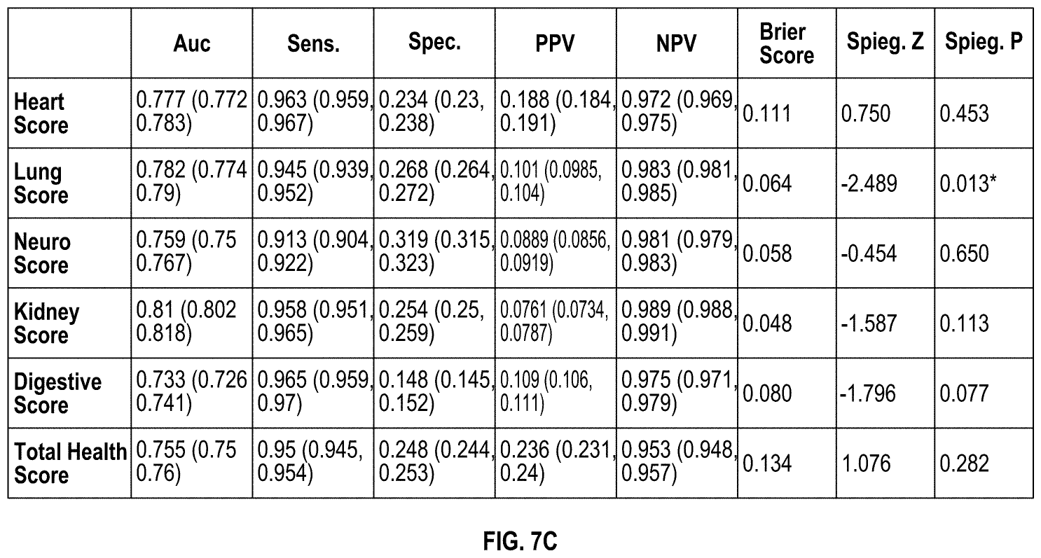

A sensitivity analysis was performed by calculating the model properties for specific age groups, namely young (under 27 years of age; table shown in FIG. 7A), adult (ages 27-64; table shown in FIG. 7B), and senior (over 64; table shown in FIG. 7C).

The AUC values were consistently high across all age groups, and decreased with age. The young group of patients showed AUCs between 0.761-0.848, adults 0.759-0.831, and seniors 0.733-0.810. Sensitivity increased with age (0.190-0.534 in youth, 0.578-0.694 in adults, and 0.914-0.984 in seniors) as well as calibration, while specificity decreased with age (0.934-0.996 in youth, 0.766-0.917 in adults, and 0.112-0.319 in seniors).

### Feature Importance

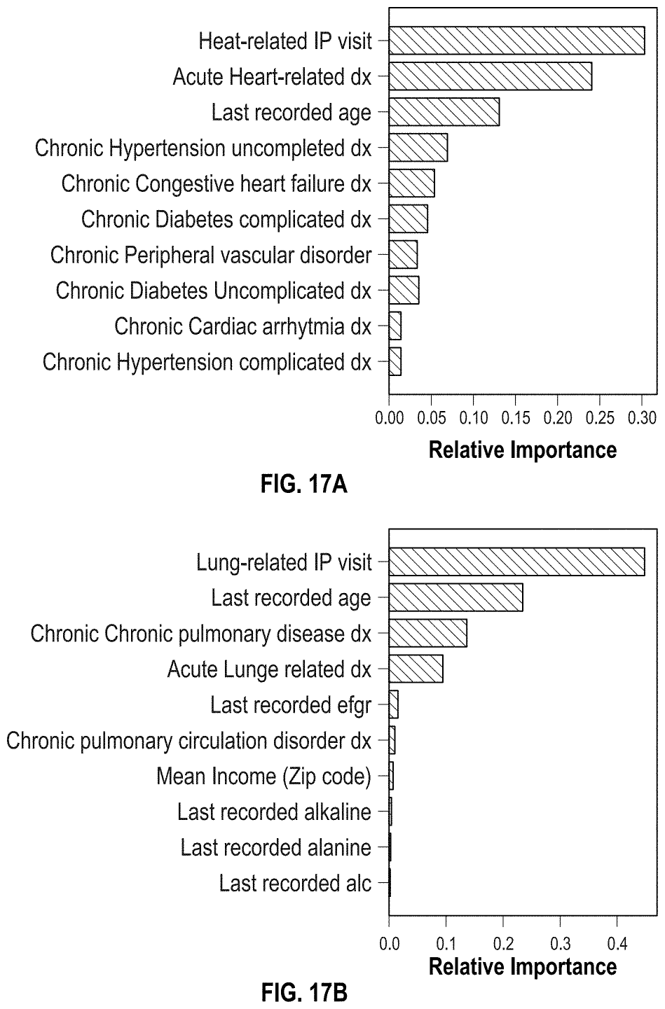

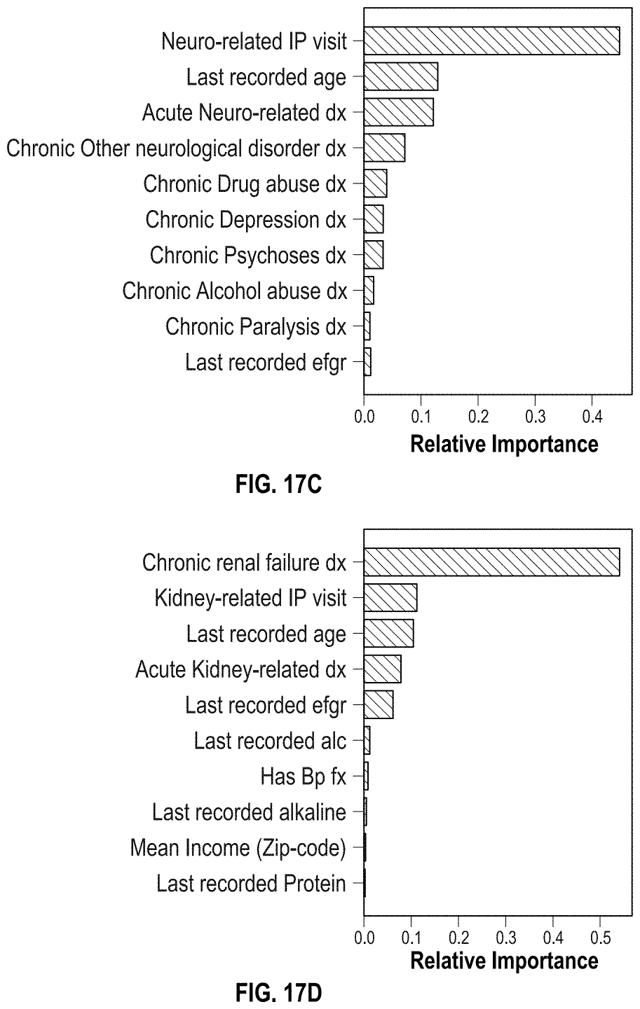

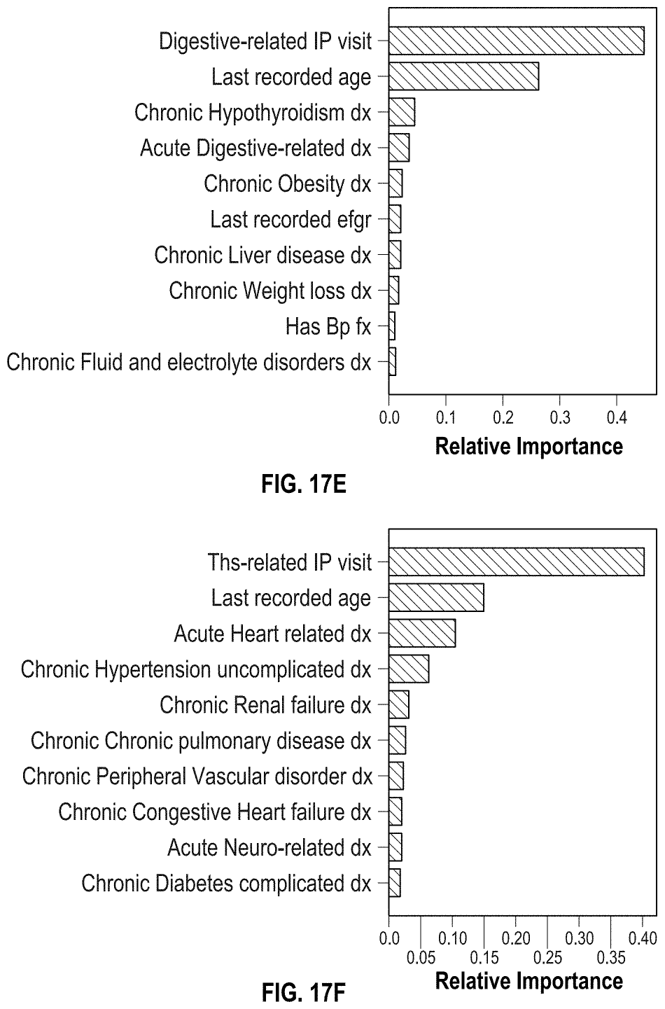

The top most important features (e.g., 10 or 15 features) of the gradient boosted classifiers for each score were selected, as shown in FIG. 17. It was found that the number of inpatient hospital visitations during the data-collection period and age are typically the two most valuable features across all risk scores; suggesting that past visits to the hospital beget future visitations, and matching our observed correlation between age and hospital visitations observed in FIG. 3. FIG. 17 shows top 10 feature importance for each risk score model. Relative importance was calculated as the normalized value of the Gini impurity. A-F refers to, in order, heart, lung, neuro, kidney, digestive, and Total-Health risk scores.

### Score Post-Processing

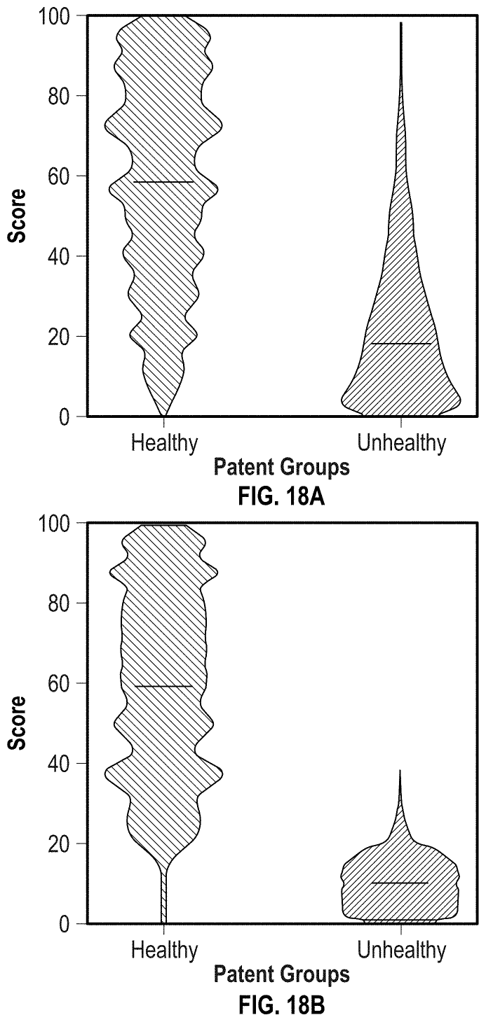

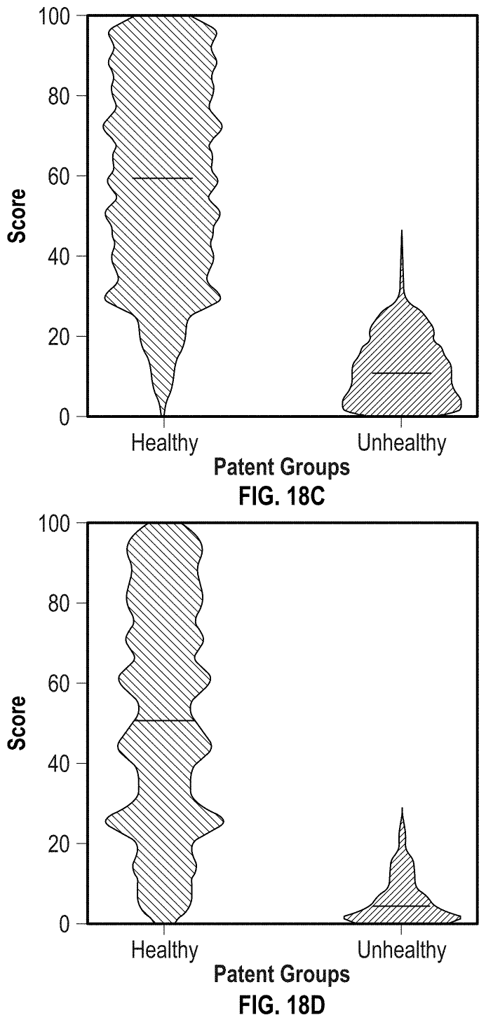

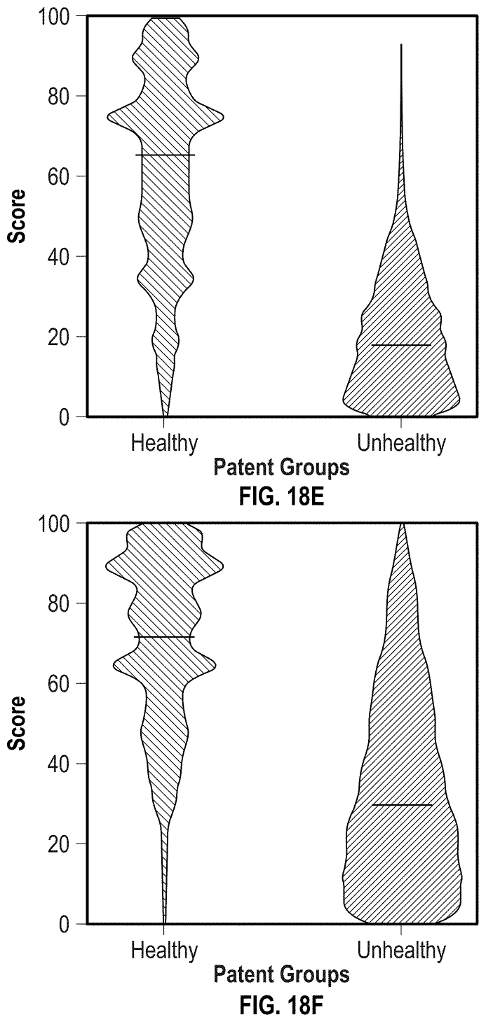

Some implementations post-process the score to better meet principles of clear medical communication, via inversion, scaling to 0-100, and normalizing by age. This process is performed for each of the six scores. The distributions of the resulting scores for healthy (no inpatient visits) and unhealthy individuals (with at least one inpatient visit) were plotted as illustrated in FIG. 18. The rescaled scores were statistically significantly higher for healthy versus unhealthy individuals across all models (p<0.01). FIG. 18 shows violin plots for predicted scores that have been post-processed to represent percentile of score within an age decade bin. Score distributions are stratified by healthy (no pre-existing score-related comorbidity) and unhealthy (at least one pre-existing score-related comorbidity). Healthy median scores were significantly higher than unhealthy scores for all models (p<0.01). A-F refers to, in order, heart, lung, neuro, kidney, digestive, and Total-Health risk scores.

### System for Calculating Multimorbidity Risk Scores for Generalizable Patient Populations

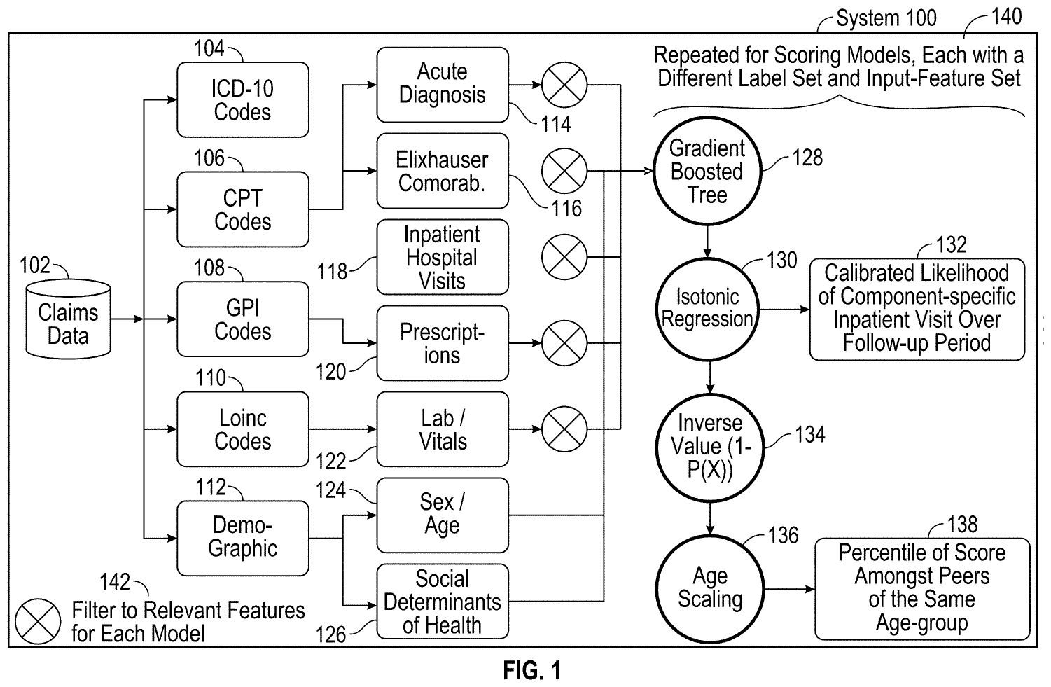

FIG. 1 shows a schematic diagram of a system 100 for calculating multimorbidity risk scores for generalizable patient populations, according to some implementations. In FIG. 1, the first column represents raw claims data 102 (e.g., claims data collected by one or more health insurance providers, such as Anthem). The second column represents individual groups of data that can be derived from the claims data. For example, this includes ICD-10 codes 104, CPT codes 106, GPI codes 108, LOINC codes 110, and/or demographics data 112, according to some implementations. The third column represents input feature/output labels that can be derived from each individual data groups (examples for how the data are selected are described below, according to some implementations). For example, the input feature or output labels include count of acute diagnosis 114, Elixhauser comorbidities 116, count of inpatient hospital visits 118, prescriptions 120, real-values of lab or vitals 122, sex or age 124, and/or social determinants of health 126, according to some implementations. The fourth column represents a filtering process 142 that is performed for the input features/output labels depending on which risk score is being calculated; the filtering process is further described below, according to some implementations. The fifth column represents the training, calibration, and age-scaling process that is performed for each clinical risk score. The process is repeated for each scoring model, each with a different label set and input feature set 140, according to some implementations. In some implementations, the process includes a gradient-boosted tree algorithm 128, isotonic regression 130 (that generates calibrated likelihood 132 of component-specific inpatient visit over follow-up period), inverse value generation 134 (that subtracts the probability from the value 1), and/or age scaling 136 (that generates percentile of score 138 amongst peers of the same age group), details of which are described below, according to some implementations.

Some implementations obtain a snapshot of overall clinical risk, or total health profile, for a patient, via organ-system-specific risk scores (CS) (e.g., five organ-specific risk scores) and a single overall risk score (sometimes called total health score or THS), using a large set of representative patients. FIG. 2A shows a table with example demographic profile of patients included in an analysis cohort, according to some implementations. FIG. 2B shows a table with example zip-code demographic profile of patients included in the analysis cohort, according to some implementations. Both FIGS. 2A and 2B illustrate examples of patient demographics, according to some implementations. FIG. 3 shows a bar chart that indicates positive correlation between percentage of patients with inpatient visits (classified as visits related to heart, lung, neuro, kidney, digestive, or Total-Health scores) and age of the patient. Various experiments showed that the THS and CS performed well at predicting patient risk (measured by the likelihood of inpatient hospital visitations), producing AUC's between 0.832-0.897 (as shown in the table in FIG. 4A and the graph in FIG. 5), and from age-specific sensitivity analysis, these AUC values remained high for all age groups, increasing with age (0.761-0.848 for young, adults 0.759-0.831, and seniors 0.733-0.810). FIG. 4A shows a table of discriminative and calibration metrics for each score in a large test set (e.g., a test set with close to 198,000 tests). Data shown as “*” correspond to models that are not well-calibrated, as indicated by two-tailed P-values and an alpha of 0.05. FIG. 4B shows Pearson R correlations between all predicted scores, for the table shown in FIG. 4A, according to some implementations. FIG. 5 shows ROC curves (with attached AUC values) that plots true positive rate against false positive rate, for each risk score (e.g., heart score, lung score, neuro score, kidney score, digestive score, and total health score), according to some implementations. Finally, the majority of the models are calibrated according to the two-tailed Spiegelhalter's p-value, and all models are calibrated according to their respective calibration plots examples of which are shown in FIG. 6. From the intercorrelation matrix of all scores, the highest correlations are noted for the cardiovascular score with the THS, which is concordant with previously published observations that cardiovascular conditions result in a significant number of inpatient stays. Experiments showed high correlation between the cardiovascular component score and the gastrointestinal component score which may be reflective of the inclusion of obesity, a well-known cardiovascular risk factor, in the gastrointestinal organ system category.

FIGS. 7A-7C show results of sensitivity analysis, according to some implementations. The tables shown in FIGS. 7A-7C show model properties and calibration statistics for patients aged less than 27, between the ages of 27 and 64, and over 64 years old, respectively. The sensitivity analysis showed that the AUCs decreased slightly with age (from 0.761-0.848 for youth to 0.733-0.810 in seniors), sensitivity increased with age (from 0.190-0.534 in youth to 0.914-0.984 in seniors), as well as the degree of calibration, and specificity decreased with age (from 0.934-0.996 in youth to 0.112-0.319 in seniors). These results reflect the trends that the presence of multiple comorbidities increase with age 47,50,54: the THS and component subscores will be less sensitive to clinical findings in younger patients as they tend to have fewer comorbidities. Conversely, specificity will decrease with age as older patients have more comorbidities which can contribute to the THS and the component scores.

Existing clinical risk scores play a vital role in health assessments and making decisions about patient management. While many EMRs have population health-based modules which can apply multiple risk scores, such as the Diabetes Risk Score or Framingham Risk score at the population level, obtaining a snapshot of a patient's complete health status would require integrating several risk scores that may not be applicable to specific patients (e.g., the CHADS2 stroke score in patients with chronic renal disease). The THS integrates diagnoses, prescriptions, procedures, and laboratory results to produce a single, scaled risk score together with organ-system Component Scores to provide a snapshot of health. This snapshot can be used for patients irrespective of age and does not require integrating several different risk scores. The score can be provided as a relative percentile on a scale from 0-100 with post-processing, making it more interpretable by patients and healthcare providers.

Although there are some limitations to the experimental study described above, these limitations are alleviated with longer or more diverse datasets or by ensuring that the data biases are not resulting in harmful model outputs. First, the population covered in the insurance claims database was drawn from zip codes that were disproportionately white, meaning that it may not be entirely representative of the US populations. Additionally, the follow-up period is two years which is shorter than the 5-10 year follow-up period of other clinical risk scores such as Framingham and the Diabetes Risk Score. Another limitation is that while the claims dataset used for this study includes populations on Medicaid and Medicare, they were likely dwarfed by those on employer plans. Thus, it is possible that the results presented here may not generalize to these likely underrepresented populations or uninsured patients. An additional potential limitation is the use of inpatient visits as a proxy for overall clinical risk. While this is a reasonable adverse health event to balance the models, due to it being positively correlated with a known indicator of unhealthiness (age) and being a tangible negative outcome patients would rather avoid, there are other options, such as a longer follow-up period with all-cause mortality as the outcome. Furthermore, the experiments assumed that the inpatient visit is related to the given Component Score, given the specified inclusion criteria. However, it should be noted that given the large and diverse cohort used in training the models, it is unlikely that this particular limitation skewed the THS and CS away from its core clinical purpose.

The results of this investigation suggest that the total health or total health score could serve as a useful, data-driven snapshot of health for healthcare professionals. Some implementations include new organ system component scores, use an expanded training set that includes more diverse populations, incorporate results from analyzing any distribution shift using more longitudinal data (e.g., using a follow-up period that is longer than two years), and/or include more forms of data (such as genomic or wearables data). Some implementations analyze the impact and potential actionability of the total health profile within care management, and introduce ways for the risk-scores to be directly actionable via therapeutics. For example, some implementations identify that the reason the heart risk score is poor is because the patient is suspected to be prediabetic. In the case that the patient receives treatment for that, some implementations adjust the score.

### Example Methods

### Experimental Design and Patient Inclusion

Some implementations use an administrative claims database (e.g., a claims database with 52 million patients provided by Anthem, an American healthcare insurance company) for a retrospective cohort study. Some implementations include patients of ages up to 90, who are enrolled in commercial, Medicare, Medicaid, and exchange plans with Anthem. Some implementations collect available diagnoses, medical procedures, prescriptions, and laboratory results from a time period (e.g., Jan. 1, 2016 through Dec. 31, 2019) for all patients who meet the selection criteria. Some implementations define a data collection period (e.g., Jan. 1, 2016 through Dec. 31, 2017), and a follow-up period (e.g., Jan. 1, 2018 through Dec. 31, 2019). Some implementations de-identify patient information by removing names, addresses, contact information, and claims identifier numbers.

Some implementations then extract diagnosis (in the form of International Classification of Disease (ICD-10) codes), medical procedures (using Current Procedural Terminology (CPT) codes), laboratory data (using Logical Observation Identifiers Names and Codes (LOINC) codes), and prescription data (derived from General Product Identifier (GPI) codes) for selected patients. In some implementations, patients who had at least one medical claim of any of these codes in each year during the time period (e.g., between 2016-2019), and had a known sex, birthdate, and zip code, are considered for inclusion in the study. Some implementations randomly select a group of patients (e.g., 992,868 patients) from the resulting patients (e.g., 14 million patients) to use as a cohort. Some implementations perform an 80:20 split on selected patients for training and testing. For example, 794,294 patients are placed in the training group and 198,574 patients are placed in in the testing group.

### Example Data Processing and Feature Extraction

Referring back to FIG. 1, in some implementations, a set of features are extracted using the data compiled for the cohort (e.g., the group of 992,868 patients). The set of features correspond to chronic diagnoses, acute diagnoses, acute procedures, prescriptions, sociodemographic information, and laboratory results or physical exam measurements, for feature extraction and modelling. To create a list of chronic conditions to include, some implementations initially extract chronic disease categories from the Elixhauser Comorbidity Index. In some implementations, a physician maps the International Classification of Diseases (ICD-10) codes corresponding to these diseases (see mapping in Supplementary Table 1). In some implementations, this data is then grouped into five organ systems (cardiovascular, respiratory, neurological, renal, gastrointestinal) which reflected the organ systems involved in the top-10 sources of mortality in the United States. Some implementations extract features for demographics, diagnoses, medical procedures, laboratory results, and/or prescriptions. This information is subsequently used to calculate the THS and the component scores, with the component scores being calculated separately for each organ system and including only information for that organ system.

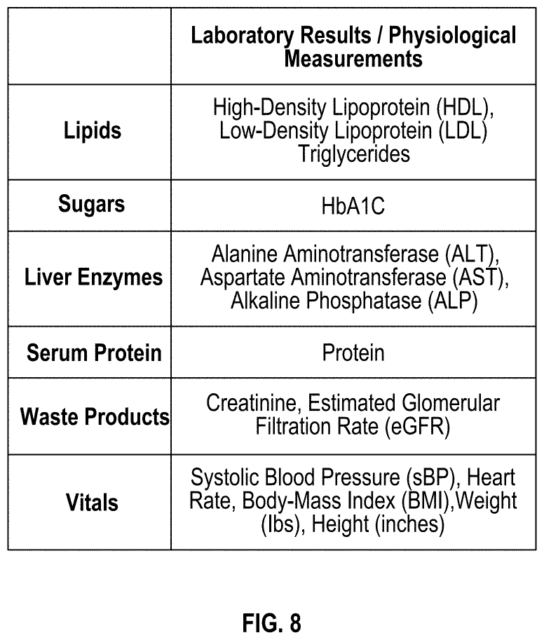

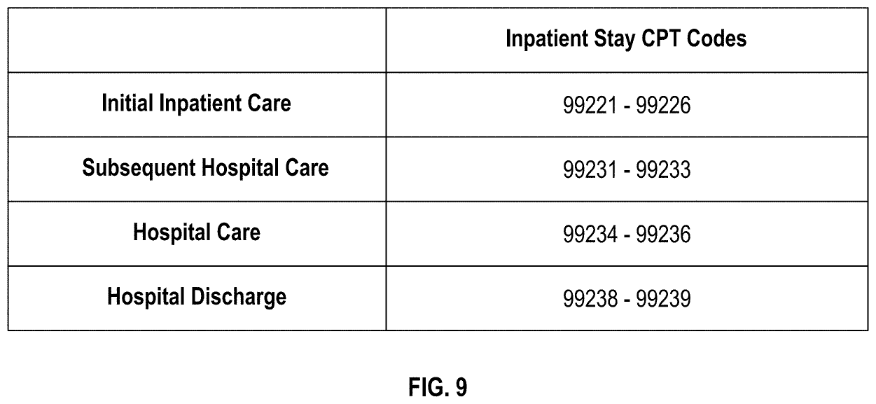

In some implementations, demographic information is extracted from a public database (e.g., the United States Census American Community Survey (ACS) for 2017) at the zip code level. In some implementations, this information includes population, household count, racial percentages for that zip code (such as African American, non-Hispanic White, Hispanic, Asian, Native American), sex percentages, and economic indicators including mean and median income. In some implementations, demographic data also includes the age and sex of the patient. In some implementations, chronic disease diagnoses are counted as the presence of a chronic disease, while acute diagnoses are counted as the number of those diagnoses in the study period, summed over the component (for instance, 3 atrial fibrillation codes and 2 acute heart failure codes during the two-year data collection period result in the number of acute heart diagnoses being 5). In some implementations, the presence of prescriptions is incorporated as binary values. In some implementations, four main groups of prescriptions were included: antihypertensives, hypoglycemics, lipid-lowering medications, and antithrombotic agents. In some implementations, laboratory data and physical measurements or vitals are included. An example list of all laboratory results or physiological measurements or vitals used in the calculation of each risk score is shown in FIG. 8, according to some implementations. In some implementations, if there are multiple laboratory/vitals collected during the data-collection period, only the most recent measurement is included. In some implementations, inpatient hospital stays are counted as the number of inpatient Current Procedural Terminology (CPT) codes that occurred during the data-collection period, subject to the same component-specific diagnostic inclusion criteria set for the CS and THS labels (discussed below). An example list of the CPT codes used to identify inpatient visits is shown in FIG. 9, according to some implementations.

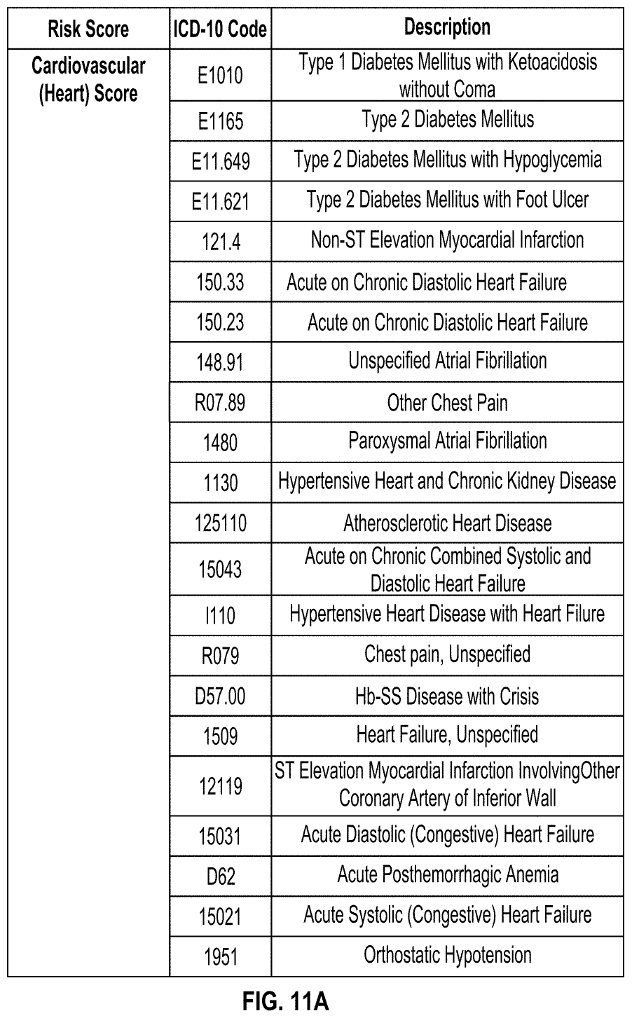

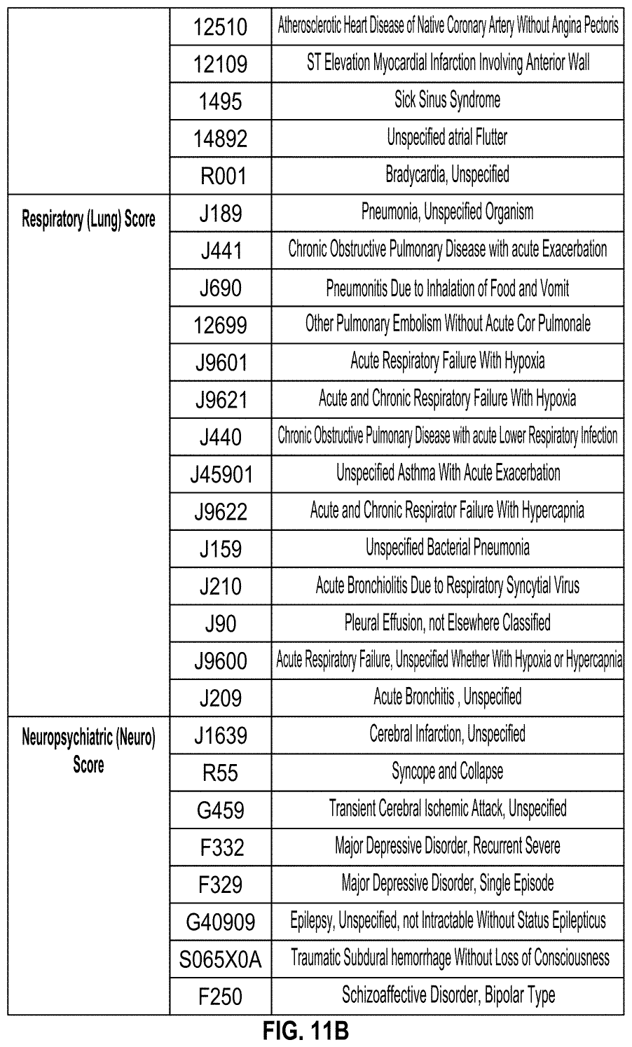

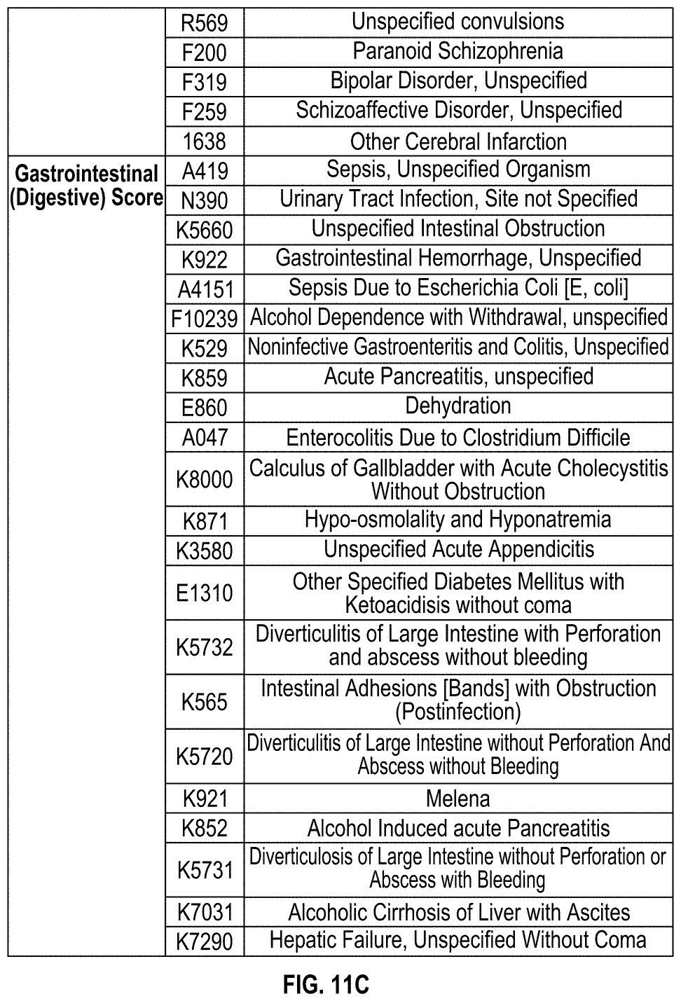

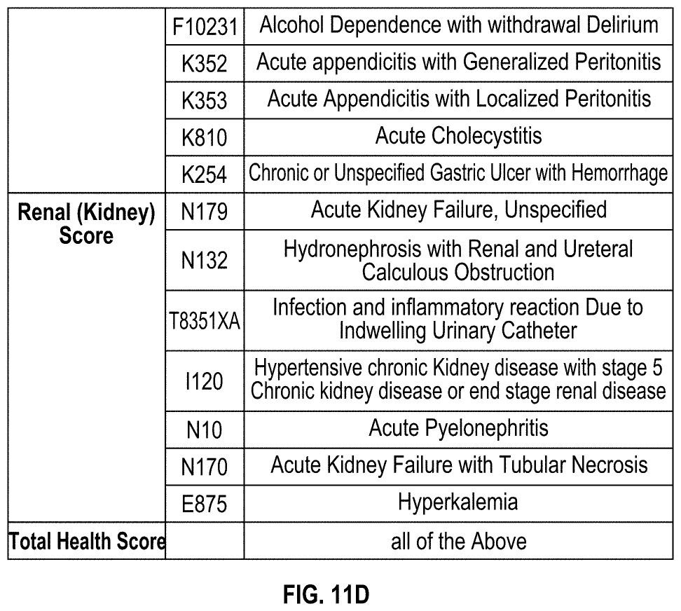

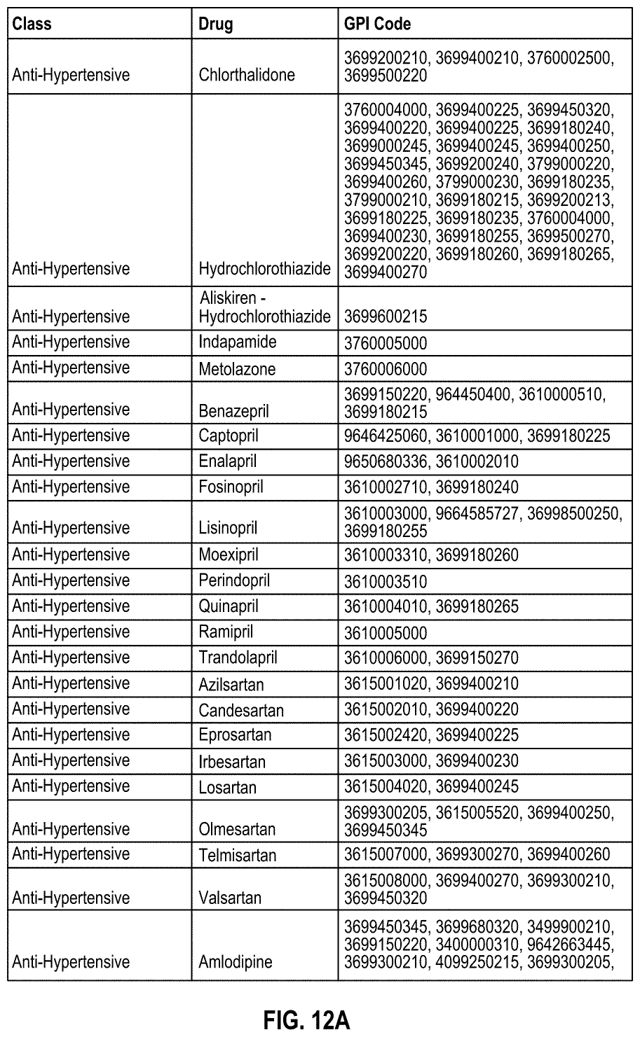

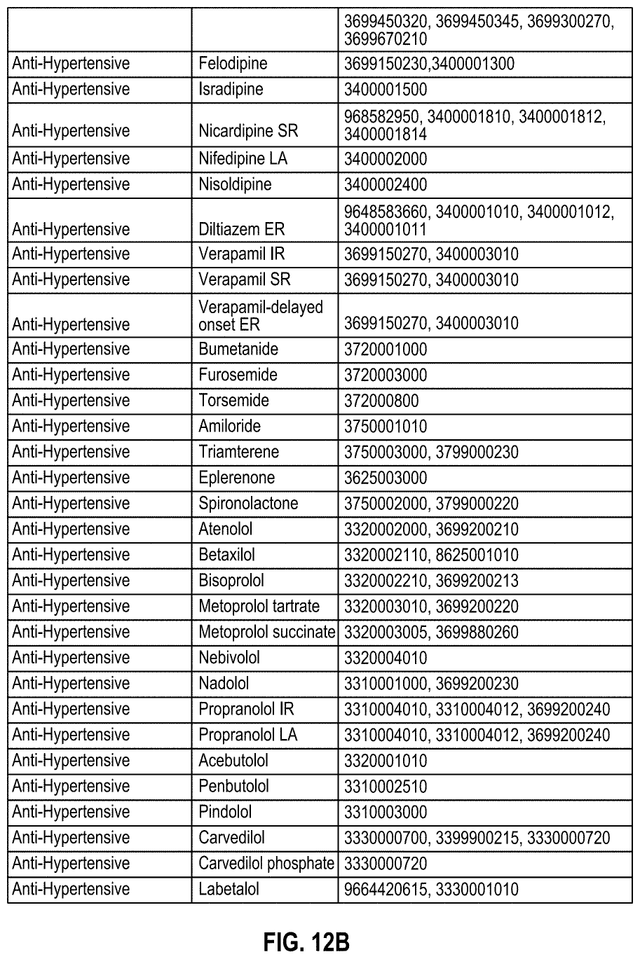

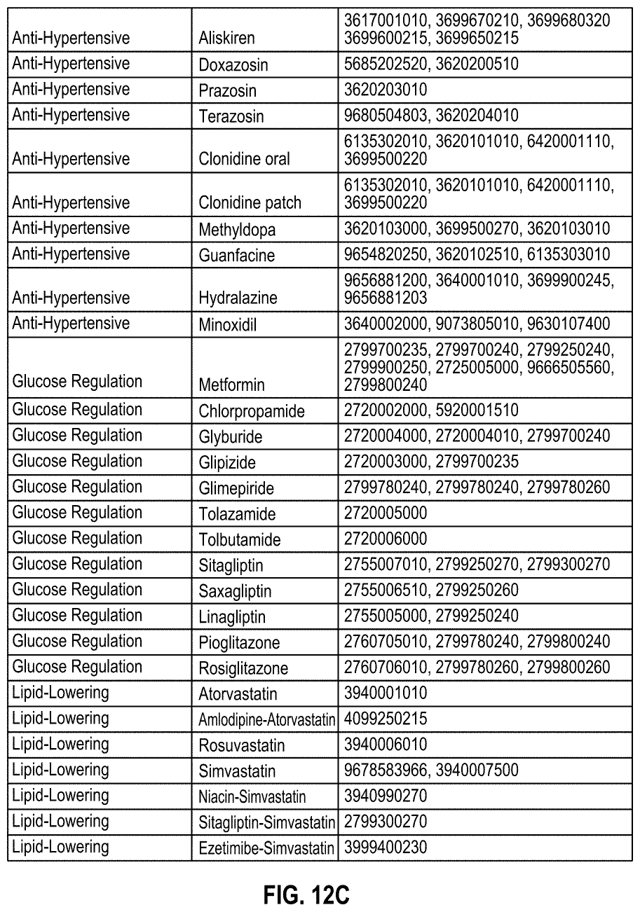

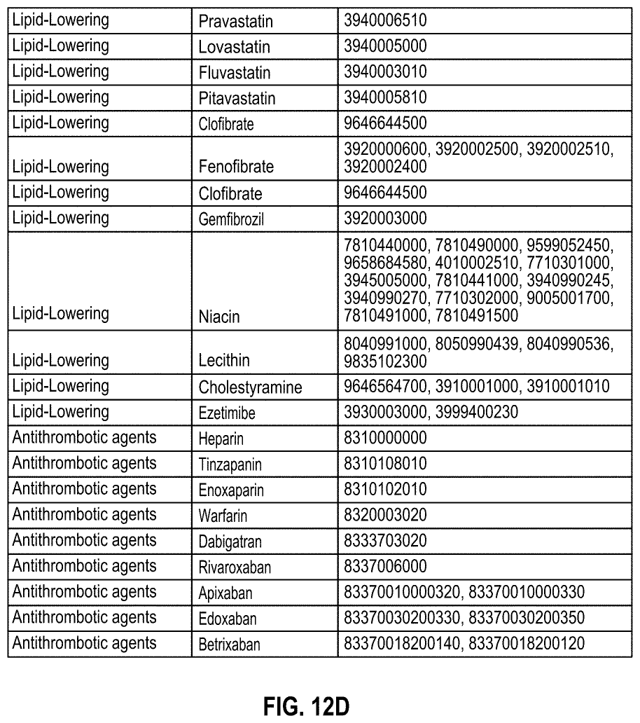

In some implementations, all demographic data and all labs or vitals are included as input features for the CS and THS model. In some implementations, for inpatient procedure features, only the IP visit count specific to the component (and the associated diagnostic inclusion criteria) are used as input to a given component model. For all other feature groups, features are stratified according to the model. FIGS. 10, 11A-11D, and 12A-12D show example tables that show a mapping of CS and THS models to their respective input features for chronic diagnoses, acute diagnoses and prescriptions, according to some implementations. FIG. 10 shows a table of chronic conditions used to derive input for each risk score. Conditions are encoded as binary variables representing the presence or absence of the corresponding chronic disease (hypertension is not included in label creation due to its commonality across all scores). FIGS. 11A-11D show a table of acute ICD Codes, stratified by CS/THS models. As input features, each row of ICD-10 codes is represented as the aggregate sum of all of the ICD-10 codes that occurred during the data-collection period, and only included as input to the model it is stratified by, according to some implementations. FIGS. 12A-12D show a table of Generic product identifier (GPI) codes corresponding to antihypertensives, glucose-lowering, lipid-lowering, and antithrombotic medications.

In some implementations, the set of input features used over all CS models are used as input for the THS model (with an exception for chronic diagnoses shown in FIG. 10).

### Example Model Labels

The presence of multiple health conditions is known to contribute to reductions in total health, reflected by functional decline and declines in the quality of life particularly in older adults, as measured by quality-adjusted life years (QALY), and can increase the risk of limitations in function. Additionally, these reductions in overall health are associated with increased hospital visits. Therefore, in some implementations, inpatient visits are selected together with diagnostic codes as a surrogate measure of overall risk, which reflects both the diagnosis of a condition and exacerbations of those conditions.

The CS label is a binary indicator referring to whether a patient had an inpatient visit within the follow-up period, given that they also had acute or chronic diagnoses within 12 months prior to the inpatient visit and within 7 days after the inpatient visit; establishing both a history of that condition and that the inpatient visit was (likely) related to that condition. In some implementations, these diagnoses are specific to each component, given by the Elixhauser comorbidities shown in the table in FIG. 10, and ICD10 codes shown in the table in FIGS. 11A-11D. For example, a possible positive label for the lung scoring model could be an inpatient hospital stay CPT code on Jun. 2, 2019, a diagnosis code corresponding to Pneumonia 3 months prior to it, and a diagnosis code corresponding to Chronic Pulmonary Disease 2 days after it. The THS label is simply the combination of all the CS labels; if a patient had any positive CS label, the THS label would be positive as well.

### Example Modeling

In some implementations, scores are calculated using a gradient-boosted tree classifier, with default hyperparameters (e.g., using the Scikit-Learn Python 3.6 package (version 0.24.1)). In some implementations, using the diagnoses, laboratory values, procedures, and prescription data as inputs and inpatient visits as binary labels, separate models are trained for each score and subsequently calibrated using an isotonic regression with 3-fold cross-validation over the training set. In 3-fold cross-validation, the original sample is randomly partitioned into three equal sized subsamples. Subsequently, of the three subsamples, a single subsample is retained as the validation data for testing the model, and the remaining subsamples are used as training data. This cross-validation process is then repeated three times, with each of the three subsamples used exactly once as the validation data. The three results are averaged to produce a single estimation. In this way, some implementations use cross-validation during the calibration process to select which calibrator to use, as cross-validation gives a very robust estimation of accuracy levels. Some implementations obtain discriminative results from the models using the optimal threshold point of the training set (given by the threshold that yielded the smallest difference between the true-positive rate and the false-positive-rate) applied to the testing set. In some implementations, missing values are mean-imputed, and all input features for each model are mean-normalized using the training data.

Some implementations use, for a baseline model, a logistic regression model with default hyperparameters using the statsmodel package (version 0.12.0). In some implementations, the baseline model is a simplified version of the CS/THS models, using a smaller and/or less user-defined set of features and/or a less complicated model. In some implementations, the baseline model is used to assert the need for the larger set of features and a more complicated model. In some implementations, the feature inputs used for the baseline model are limited to Elixhauser Comorbidities, defined by the mapping of component score/THS shown in the table in FIG. 10.

Some implementations use a different model, such as logistic regression, Support Vector Machine (SVM), or deep learning. Some implementations use similar labels as described above, but using a different set of ICD-10 codes for each condition as inclusion criteria. Some implementations use the same model or label as described above, but alter the exact feature inputs used in each risk score model.

### Example Validation and Sensitivity Analysis

To assess the discriminative performance of each model in the THS, an experiment was conducted to generate receiver-operator curves (ROCs) and calculate the area under the curve (AUC), sensitivity, specificity, positive predictive value (PPV), and negative predictive value (NPV) using scikit-learn for the test-set of 198,574 patients. The precision-recall curve was plotted for all scores on the test-set, as shown in FIG. 13. All confidence intervals for the discriminative metrics were generated using 500 bootstrap samples of 20,000 from the test dataset. To assess calibration performance, Brier Scores and Spiegelhalter's Z-score were calculated using scikit-learn and a custom implementation, respectively. Calibration plots were calculated with a uniform bin size of 10. The models were judged for calibration based on the models had low Brier Scores, low Spiegelhalter's Z-score, high (above 0.05) Spiegelhalter's p values, and a calibration plot with a slope close to 1. Algorithm performance can be largely assessed via AUC (as it represents a comprehensive measure of the true-positive-rate and false-positive-rate tradeoff), but, due to the severe class imbalance, AUC may give overly optimistic results so sensitivity and specificity should also be noted for analyzing algorithm performance. The clinical utility of the model can be assessed via PPV, NPV, sensitivity, and calibration metrics (as it gives a clear idea of these scores can be used to identify sick patients, avoid alarm fatigue, and be interpreted as a probabilistic likelihood).

To perform sensitivity analysis of all of the models, patients were classified into three age ranges: youth (<27 years old), adults (27-64 years old inclusive), and senior (>64 years old), and quantitative discrimination and calibration metrics were calculated for each group. The distributions of the scores were plotted for healthy (no comorbidities) and unhealthy (1+ comorbidities) patients to assess expected trends. Finally, a correlation matrix between all predicted scores was created to analyze relationships, which was generated using Python Pandas (version 0.25.3).

### Example Feature Importance Calculation

Some implementations derive feature importances from the trained, gradient-boosted trees from their normalized Gini importance/information gain (e.g., using scikit-learn). Due to the isotonic calibration performed on the gradient boosted classifier and the cross-validation size of three, there are three different models with unique (but likely similar) feature rankings for each score prediction. In order to report a single set of ranks for a given model, some implementations average relative feature importance of each feature of these three models.

### Example Score Post-Processing

Score post-processing includes inversion, re-scaling, and normalization by age, according to some implementations. Outputs of the calibrated, gradient-boosted tree model represent calibrated likelihoods of an inpatient visitation from [0, 1], where 1 can be considered as a 100% chance that a patient will have an inpatient visitation during the follow-up period. For the inversion step, some implementations subtract the likelihood from 1. Some implementations multiply the resulting number by 100 to scale the number between 0 to 100. To normalize by age, the score is replaced by the percentile it is amongst patients of the same age-group (e.g., ages 0-10, 10-20). This processed value is interpreted as a patients percentile amongst those in a similar age-bucket, where higher value is better. A graphical representation of this process is shown in the right-side of FIG. 1, according to some implementations.

Some implementations use the total health score to assess patient risk, explain that risk to both patients and clinicians in an actionable manner, and allocate healthcare resources accordingly. Because the scores are calculated passively or ahead of time, clinicians or healthcare professions do not need to ask questions to patient at the time of service. Furthermore, the calculated scores or models are applicable for a vast general population of patients, and for different age groups.

FIG. 14 shows a table of baseline, logistic regression metric metrics for each score in a test set (e.g., a test set with close to 198,000 tests), according to some implementations. For this example, only component-specific Elixhauser Comorbidities shown in FIG. 10 are used as input and otherwise identical labels are used. In the table shown, “*” indicates models that are not well-calibrated given the indicated two-tailed P-values and an alpha of 0.05.

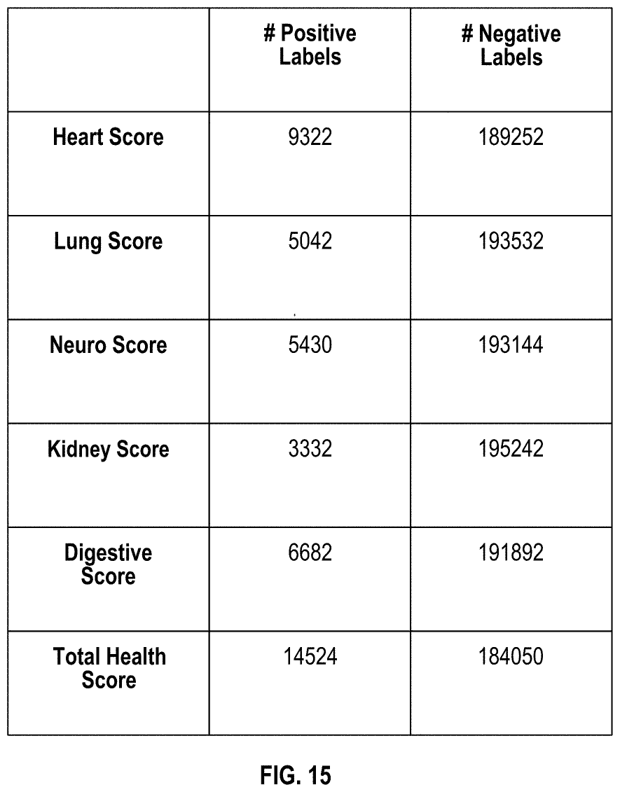

FIG. 15 shows a table of positive and negative label counts corresponding to each of the individual scores for a large data set (e.g., a test set with close to 198,000 tests). Positive labels refer to relevant inpatient-visitations for each score.

### Example of Age Score Conversion

As an example of the process of age score conversion, suppose a 33 year-olds patient's THS is 0.22, representing a calibrated probability of 22% of a patient having any of the predefined medical events in the next two years. Some implementations convert this score to 78. Subsequently, some implementations calculate the percentile of this patient against those in the same decade age-range as them (in this example, against patients between 30 and 40). Suppose further that this score of 78 directly translates to the 25th percentile of all other patients in the patient's age group, so the final THS score would be 25. In this light, the patient can be more easily informed that they are drastically unhealthy for their age group and take precautionary measures by looking at which of their component scores is causing the unhealth.

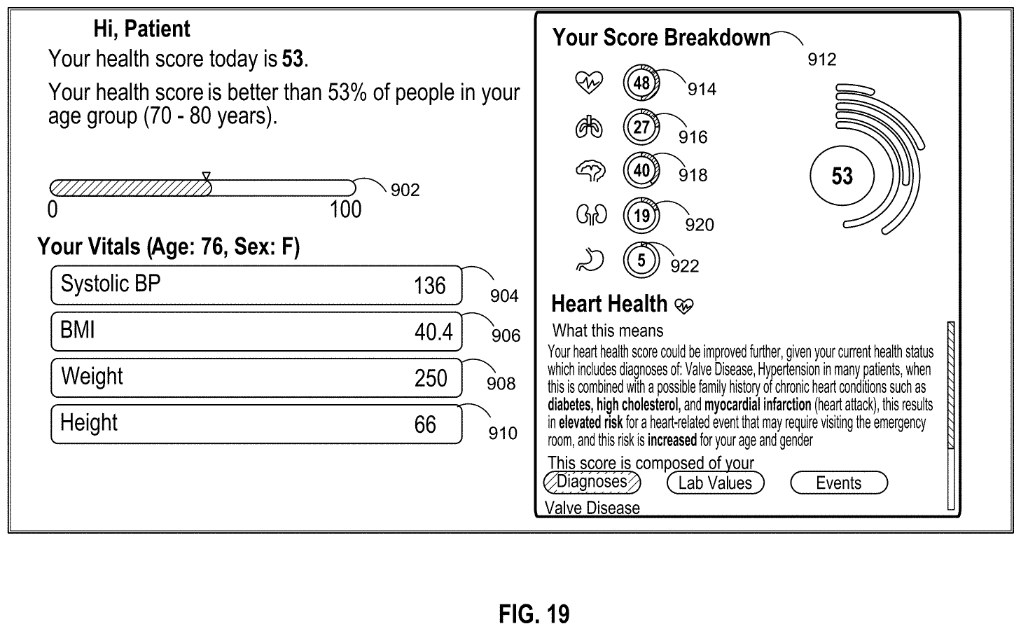

FIG. 19 shows an example interface with health breakdown. In some embodiments, FIG. 19 may be an example patient interface depicting a patient's health score. The example interface shown describes a 76 year-old female with a health score 902 of 53. In some embodiments, a health score 902 of 53 indicates that the patient has a health score 902 better than 53% in a similar age group (e.g. 70-80 years of age). The example interface also shows vitals of the patient including a systolic blood pressure (BP) 904 of 136, body mass index (BMI) 906 of 40.4, weight 908 of 250 lbs and a height 910 of 66 inches. The overall health score 902 may be broken down further as shown in score breakdown 912. Score breakdown 912 may take into account heart health 914, lung health 916, brain health 918, kidney health 920 and digestive health 922.

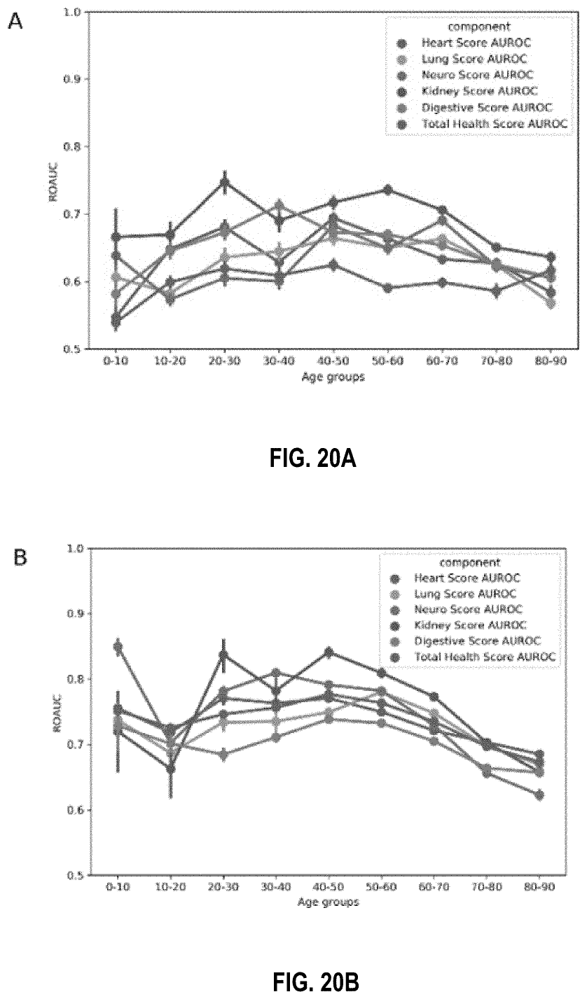

FIGS. 20A and 20B depict area under receiver-operation curve (AUROC) variations across decade age-groups and sexes for each of the six risk scores, according to some implementations. FIG. 20A is limited to male patients and FIG. 20B is limited to femal patients. All models demonstrated relatively consistent performance, with some exceptions, across patients of different ages and gender. Predictive performance showed, in some implementations, a weak positive correlation to disease-burden.

Although some of various drawings illustrate a number of logical stages in a particular order, stages that are not order dependent may be reordered and other stages may be combined or broken out. While some reordering or other groupings are specifically mentioned, others will be obvious to those of ordinary skill in the art, so the ordering and groupings presented herein are not an exhaustive list of alternatives. Moreover, it should be recognized that the stages could be implemented in hardware, firmware, software or any combination thereof.

The foregoing description, for purpose of explanation, has been described with reference to specific implementations. However, the illustrative discussions above are not intended to be exhaustive or to limit the scope of the claims to the precise forms disclosed. Many modifications and variations are possible in view of the above teachings. The implementations were chosen in order to best explain the principles underlying the claims and their practical applications, to thereby enable others skilled in the art to best use the implementations with various modifications as are suited to the particular uses contemplated.

What is claimed is:

1. A computer-implemented method of generating health care plans for patients, the method comprising:
at a computing device coupled to one or more memory units each operable to store at least one program; and one or more servers having at least one processor communicatively coupled to the one or more memory units, in which the at least one program, when executed by the at least one processor, causes the at least one processor to perform:
extracting data items from age-agnostic medical claims data for a plurality of patients;
for each organ-system-specific health condition of a plurality of organ-system-specific health conditions for a respective patient, wherein the plurality of organ-system-specific health conditions includes cardiovascular, respiratory, neuropsychiatric, renal, and gastrointestinal conditions:
aggregating one or more of the data items into one or more feature sets based at least on a data item type and a set of rules; and
applying one or more machine learning models to the one or more feature sets to predict a respective risk score for the respective health condition for a respective patient, wherein the one or more machine learning models were previously trained by performing risk classification analysis on data items from the age-agnostic medical claims data for the plurality of patients to calculate organ-system-specific risk score representing health risks for a specific organ-system;
computing a total health score based on the predicted respective risk score for each health condition for the respective patient; and
generating a report that indicates a health care plan for the respective patient based on the total health score in relation to a particular age group, wherein generating the report includes concurrently displaying the total health score and a breakdown of the total health score in terms of the respective score for each organ-system-specific health condition, a comparison of the total health score of the respective patient to other patients in same age group as the respective patient, vitals, and/or data used to compute the total health score, in addition to a health care plan for alleviating at least some of the organ-system-specific health conditions.

2. The method of claim 1, wherein the respective risk score represents the likelihood of inpatient hospital visits over a predetermined future time period for the respective health condition.

3. The method of claim 1, wherein the one or more machine learning models include a respective machine learning model for each health condition of the plurality of health conditions, the method further comprising:
applying the respective machine learning model for the respective health condition to the one or more feature sets to predict the respective risk score for the respective health condition for a respective patient.

4. The method of claim 1, wherein the medical claims data includes demographic information, diagnostic codes, laboratory results, prescriptions, and medical procedural data.

5. The method of claim 1, wherein the one or more machine learning models include a respective gradient boosted classifier for each health condition.

6. The method of claim 5, further comprising:
aggregating the one or more of the data items into one or more feature sets further based on selecting a predetermined number of features of the respective gradient boosted classifier for the respective health condition.

7. The method of claim 6, wherein the predetermined number of features includes number of inpatient hospital visitations during the data-collection period and

8. The method of claim 1, further comprising:
performing steps of inversion, scaling to 0-100, and normalization by age, on the respective score, for generating the report.

9. The method of claim 8, wherein the one or more machine learning models includes a gradient-boosted tree model that outputs calibrated likelihoods of an inpatient visitation between [0, 1], where 1 represents a 100% chance that a patient will have an inpatient visitation during a predetermined follow-up period, and wherein the inversion comprises subtracting the likelihood from 1, scaling includes multiplying result of the inversion by 100, and normalization by age includes calculating percentile amongst patients of a predetermined age group.

10. The method of claim 1, further comprising:
calculating correlation between the respective score for each health condition and the total health score, while generating the report.

11. The method of claim 1, wherein the one or more machine learning models include a gradient-boosted tree classifier that is trained using a training dataset that includes diagnoses, laboratory values, procedures, and prescription data as inputs and inpatient visits as binary labels, and calibrated using an isotonic regression with 3-fold cross-validation over the training dataset.

12. A system for generating health care plans for patients, comprising:
one or more processors;
memory; and
one or more programs stored in the memory, wherein the one or more programs are configured for execution by the one or more processors and include instructions for:
extracting data items from age-agnostic medical claims data for a plurality of patients;
for each organ-system-specific health condition of a plurality of organ-system-specific health conditions for a respective patient, wherein the plurality of organ-system-specific health conditions includes cardiovascular, respiratory, neuropsychiatric, renal, and gastrointestinal conditions:
aggregating one or more of the data items into one or more feature sets based at least on a data item type and a set of rules; and
applying one or more machine learning models to the one or more feature sets to predict a respective risk score for the respective health condition for a respective patient, wherein the one or more machine learning models were previously trained by performing risk classification analysis on data items from the age-agnostic medical claims data for the plurality of patients to calculate organ-system-specific risk score representing health risks for a specific organ-system;
computing a total health score based on the predicted respective risk score for each health condition for the respective patient; and
generating a report that indicates a health care plan for the respective patient based on the total health score in relation to a particular age group, wherein generating the report includes concurrently displaying the total health score and a breakdown of the total health score in terms of the respective score for each organ-system-specific health condition, a comparison of the total health score of the respective patient to other patients in same age group as the respective patient, vitals, and/or data used to compute the total health score, in addition to a health care plan for alleviating at least some of the organ-system-specific health conditions.
